(19) United States
(12) Patent Application Publication
(10) Pub. No.: US 2023/0002508 A1
(43) Pub. Date: Jan. 5, 2023

(54) USE OF AN ANTI-FACTOR XII ANTIBODY FOR THE TREATMENT OR PREVENTION OF HEREDITARY ANGIOEDEMA

(71) Applicant: CSL INNOVATION PTY LTD, Parkville (AU)

(72) Inventors: lngo PRAGST, Edertal (DE); Dipti PAWASKAR, Philadelphia, PA (US); Theresa YURASZECK, Malvern, PA (US); Ying ZHANG, Chesterbrook, PA (US)

(21) Appl. No.: 17/781,995

(22) PCT Filed: Dec. 3, 2020
(86) PCT No.: PCT/AU2020/051321

§ 371 Date: Jun. 2, 2022

Related U.S. Application Data
(60) Provisional application No. 63/093,975, filed on Oct. 20, 2020.
(60) Provisional application No. 62/943,117, filed on Dec. 3, 2019.

(51) Int. Cl. C07K 16/36; A61P 7/00

(52) U.S. Cl. CPC C07K 16/36; A61P 7/00; C07K 2317/565; C07K 2317/21; C07K 2317/76

(57) ABSTRACT

The present disclosure relates to methods of treating or preventing hereditary angioedema (HAE) in a subject in need thereof by subcutaneously administering to the subject an effective amount an anti-FXII antibody comprising (i) a V_{H} comprising a CDRH1 comprising a sequence set forth in SEQ ID NO:1; a CDRH2 comprising a sequence set forth in SEQ ID NO:2; and a CDRH3 comprising a sequence set forth in SEQ ID NO:3; and (ii) a V_{L} comprising a CDRL1 comprising a sequence set forth in SEQ ID NO:4; a CDRL2 comprising a sequence set forth in SEQ ID NO:5; and a CDRL3 comprising a sequence set forth in SEQ ID NO:6.

The present invention relates to an anti-FXII antibody for use in a method of treating or preventing hereditary angioedema (HAE) in a subject, wherein the antibody is administered subcutaneously to the subject.

### BACKGROUND

Factor XII (Hageman Factor, FXII) is a serum glycoprotein with a molecular weight of about 80 kDa. Besides an autoactivation by exposure to negatively charged surfaces, factor XII is additionally activated by kallikrein by proteolytic cleavage to form alpha-factor XIIa, which is then further converted, for example by trypsin, into beta-factor XIIa (FXIIa-β). Alpha-factor XIIa is composed of the N-terminal heavy chain of about 50 kDa, which contains the contact binding domain, and the C-terminal light chain of about 28 kDa, which contains the catalytic center. The heavy and light chains are connected by a disulfide bond. FXIIa-β is an active form of FXII of about 30 kDa, consisting of the complete light chain and a 2000 Da fragment of the heavy chain linked by a disulfide bond.

Hereditary angioedema is a rare genetic disorder classified into 3 disease types [Rosen, et al. 1965, Science 148; 3672:957-8; Bork, et al. 2000, Lancet, 356; 9225:213-7], including HAE type 1, HAE type 2, and HAE with normalC1-esterase inhibitor (nC1-INH). HAE type 1 and type 2 are caused by mutations of the SERPING1 gene, and are characterized by a quantitative decrease in C1-esterase inhibitor (C1-INH) plasma concentrations (type 1) and dysfunctional C1-INH present in normal plasma concentrations (type 2) [Zuraw, et al. 2010, N Engl J Med, 363; 6:513-22; Cicardi, et al. 2014, Allergy, 69; 5:602-16]. Together, HAE type 1 and type 2 are grouped as HAE with C1-INH deficiency (C1-INH HAE). C1-esterase inhibitor is a serine protease inhibitor that regulates the generation of BK by the plasma contact system, and is the major inhibitor of a number of plasma contact system proteases including FXII and kallikrein [Davis, et al. 2010, Thromb Haemost, 104; 5:886-93]. Excessive BK formation due to pathological activation of the factor XII (FXII)-driven plasma contact system is a consistent finding in acute episodes of HAE [Björkqvist, et al. 2013, Thromb Haemost, 110; 3:399-407] (see FIG. 1).

HAE with normal C1-INH (nC1-INH) is an inherited disorder not associated with C1-INH deficiency but missense mutations, deletions or insertions of base pairs of the FXII gene [Cicardi, et al. 2014, Allergy, 69; 5:602-16], a missense mutation of the plasminogen gene [Bork, et al. 2018, Allergy, 73; 2:442-50], [Dewald, 2018, Biochem Biophys Res Commun, 498; 1:193-8], or caused by an unknown genetic defect [Cicardi, et al. 2014, Allergy, 69; 5:602-16].

Clinically, HAE attacks occurring in patients with HAE are characterized by local swelling of the skin (ie, edema of the extremities, facial edema, and edema of the genitals), abdominal pain, and, occasionally, life-threatening attacks of laryngeal edema [Bork, 2008, Exp Rev Clin Immunol, 4; 1:13-20]. The estimated prevalence of C1-INH HAE is commonly reported as 1:50,000, while the prevalence of nC1-INH HAE is unknown [Cicardi, et al. 2010, N Engl J Med, 363; 6:523-31; Nasr, et al. 2016, Exp Rev Clin Immunol, 12; 1:19-31].

Current treatment options for HAE can be subdivided into the acute treatment of attacks and prophylaxis. Acute and prophylactic treatments for HAE are based on blocking BK production through targeting different proteins in the kallikrein-kinin pathway. The treatment of choice in the event of an acute HAE attack is the rapid intravenous (IV) administration of C1-INH concentrate [Bork, 2008, Exp Rev Clin Immunol, 4; 1:13-20; Gompels, et al. 2005, Clin Exp Immunol, 139; 3:379-94; Longhurst, 2005, Int J Clin Practice, 59; 5:594-9]. Recently, compounds including a kallikrein inhibitor and a BK receptor antagonist have been added to the spectrum of medications available to treat acute HAE attacks [Cicardi, et al. 2010, N Engl J Med, 363; 6:532-41; Cicardi, et al. 2010, N Engl J Med, 363; 6:523-31].

Currently approved C1-INH concentrates to treat acute HAE attacks intravenously are plasma-derived Berinert® and recombinant Ruconest®. Alternatively, Kalbitor® (icatibant), a kallikrein inhibitor, or Firazyr® (ecallantide), a bradykinin B2 receptor antagonist, can be administered subcutaneously in case of an acute HAE attack. The treatment options for prophylactic treatment of HAE are limited to plasma-derived Cinryze® (IV) HAEGARDA/Berinert 2000/3000 (SC). Most recently, the kallikrein antibody product Takhzyro® (lanadelumab, SC) has been approved as an alternative option for prophylaxis.

Despite the existing treatment options for acute HAE attacks and prophylactic treatment of HAE there is still an area of unmet medical need in particular in the field of prophylaxis. Limitations of current prophylactic therapies are an unfavorable side effect profile (attenuated androgens), a lack of effect (antifibrinolytics), or the frequency of administration (IV or SC C1-INH). Additionally, plasma-derived C1-INH products may experience from time to time supply issues and for that reason alternative treatment options are still required.

WO 2013/014092 and WO 2017/015431 disclose various anti-FXII antibodies and their use in the treatment of various diseases including but not limited to HAE. No in-vivo experimental data, safety data in patients or any data on clinical studies are provided with respect to HAE.

WO 2017/173494 discloses further anti-FXII antibodies including but not limited to the antibodies used in the context of the present invention. HAE is not mentioned in WO 2017/173494.

Finally, it remained unknown whether an anti-Factor XII mAb would be effective in the treatment or prevention of HAE.

Overall, although emerging therapies are providing improved prophylactic clinical outcomes, there is a need for further modalities in the prophylactic management of HAE, especially those targeting novel pharmacological pathways.

### SUMMARY OF THE INVENTION

The present invention relates to an anti-FXII antibody comprising
(i) a V_{H} comprising a CDRH1 comprising a sequence set forth in SEQ ID NO:1; a CDRH2 comprising a sequence set forth in SEQ ID NO:2; and a CDRH3 comprising a sequence set forth in SEQ ID NO:3; and
(ii) a V_{L} comprising a CDRL1 comprising a sequence set forth in SEQ ID NO:4; a CDRL2 comprising a sequence set forth in SEQ ID NO:5; and a CDRL3 comprising a sequence set forth in SEQ ID NO:6;
   for use in a method of treating or preventing hereditary angioedema (HAE) in a subject, wherein the antibody is administered subcutaneously to the subject.

In a preferred embodiment, the anti-FXII antibody comprises a V_{H} comprising a sequence set forth in SEQ ID NO:7 and a V_{L} comprising a sequence set forth in SEQ ID NO:8.

In a preferred embodiment, the anti-FXII antibody is an IgG, preferably an IgG4 antibody.

In a preferred embodiment, the anti-FXII antibody comprises a heavy chain sequence set forth in SEQ ID NO:9 and a light chain sequence set forth in SEQ ID NO:10.

In a preferred embodiment, the heavy chain comprises an additional lysine linked to the last amino acid of SEQ ID NO:9.

In a preferred embodiment, the anti-FXII antibody is administered in an amount to maintain a concentration of the antibody of at least 5 μg/mL between two subsequent administrations of the antibody.

In a preferred embodiment, the antibody is administered at a dosage of 70 mg to 700 mg once every 1-3 months, preferably once every 1-2 months.

In a preferred embodiment, the antibody is administered at a dosage of 150 mg to 250 mg, preferably 170 mg to 220 mg, more preferably 200 mg.

In a preferred embodiment, the antibody is administered at a dosage of 50 mg to 150 mg, preferably 70 mg to 130 mg, more preferably 100 mg.

In a preferred embodiment, the antibody is administered every 1-2 months, preferably once every 1 month.

In a preferred embodiment, the subject is a human patient having, suspected of having or at risk for HAE.

In a preferred embodiment, the method includes an administration of a loading dose of the anti-FXII antibody.

In a preferred embodiment, the administration of a loading dose is an intravenous administration of the anti-FXII antibody at a dosage of between 30 mg and 400 mg, preferably between 100 and 300 mg, more preferably about 200 mg.

In a preferred embodiment, the administration of a loading dose is a subcutaneous administration of the anti-FXII antibody at a dosage of between 70 mg and 700 mg, preferably between 200 and 500 mg, more preferably about 400 mg.

In a preferred embodiment, the anti-FXII antibody is only administered subcutaneously to the subject.

In a preferred embodiment, the administration of the anti-FXII antibody reduces the risk of an HAE attack, preferably by more than 85%, more preferably more than 90% and even more preferably by more than 95% or more than 98%.

### DETAILED DESCRIPTION OF THE INVENTION

According to the invention, an “anti-FXII antibody” binds to and inhibits the activated form of FXII, namely FXIIa-beta (beta-factor XIIa), but also binds to FXII and FXIIa (alpha-factor XIIa).

“Antibody” in its broadest sense is a polypeptide comprising an immunoglobulin variable region which specifically recognizes an epitope on an antigen. The term “antibody” also includes an antibody fragment that maintains the ability to bind to FXIIa and FXII. Preferred antigen binding fragments are an Fab fragment, an Fab′ fragment, an F(ab′)₂ fragment, an Fv fragment, a single chain antibody, a single chain Fv fragment, a disulfide stabilized Fv protein, or a dimer of a single chain Fv fragment. Antibodies also included in the invention are a chimeric antibody, a humanized antibody, a murinized antibody or a bispecific antibody. Methods for producing these fragments and antibodies are well known in the art (see for example, Harlow & Lane: Antibodies, A Laboratory Manual, Cold Spring Harbor Laboratory, 1988).

Antibodies are usually comprised of two identical heavy chains and two identical light chains, each of which has a variable region at its N-terminus (V_{H} and V_{L} region). Usually a V_{H} and a V_{L} region will combine to form the antigen binding site. However, single domain antibodies, where only one variable region is present and binds to the antigen, have also been described.

Typically, an antibody contains two heavy and two light chains, connected by disulfide bonds. There are 5 major isotypes of antibodies (IgG, IgM, IgE, IgA, IgD), some of which occur as multimers of the basic antibody structure. The isotype is determined by the constant region of the heavy chains. There are two types of light chains, lambda and kappa.

The term “antibody” as used herein includes intact antibodies (also known as full length antibodies, or antibodies which comprise both heavy and light chain variable and constant domains), as well as variants and portions thereof that retain antigen binding. This includes fragments of antibodies such as Fab fragments, F(ab′)₂ fragments, Fab′ fragments, single chain Fv fragments, or disulfide-stabilized Fv fragments. Thus, the term “antibody or antigen-binding fragment thereof” when use herein is only precautionary, the term “antibody” alone is already intended to cover the antibody and antigen-binding fragments thereof.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

Each heavy and light chain consists of a variable region and a constant region. The variable regions contain framework residues and hypervariable regions, which are also called complementarity determining regions or CDRs.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs.

As used herein, the term “complementarity determining regions” (syn. CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. The extent of the framework residues and CDRs is determined according to Kabat; the Kabat database is available online (Kabat E A, Wu T T, Perry H M, Gottesman K S, Foeller C (1991) Sequences of proteins of immunological interest, 5^{th} edn. U.S. Department of Health and Human services, NIH, Bethesda, Md.). The CDR regions are important in binding to the epitope and therefore determine the specificity of the antibody.

“Framework regions” (FRs) are those variable domain residues other than the CDR residues.

A “monoclonal antibody” is an antibody produced by a single clone of B lymphocytes, or by a cell line engineered to express a single antibody.

A “chimeric antibody” is an antibody with the variable regions from one species grafted onto the constant regions from a different species. A “humanized” antibody is an antibody where CDR regions from a different species, e.g. a mouse monoclonal antibody, are grafted into the framework of a human antibody. Analogously, a “murinized” antibody is an antibody where the CDR regions from a different species, e.g. a human monoclonal antibody, are grafted into the framework of a mouse antibody. A human antibody is an antibody that is wholly derived from human, i.e. human CDRs in a human framework and any constant region suitable for administration to a human.

A “germlined” antibody is an antibody where somatic mutations that introduced changes into the framework residues are reversed to the original sequence present in the genome.

“Antigen binding fragment” refers to any fragment of an antibody that retains the ability to specifically bind the epitope of the antigen that the antibody binds to. These include but are not limited to Fab, F(ab′)₂, or single chain Fv fragments.

“Binding affinity” refers to the affinity of the antibody to its antigen. It can be measured by a variety of techniques, e.g. surface plasmon resonance based technology (BiaCore®).

“Epitope” is the antigenic determinant, it is defined by the residues or particular chemical structures that the antibody makes contact with on the antigen.

“Sequence identity” relates to the similarity of amino acid sequences. The best possible alignment of two sequences is prepared, and the sequence identity is determined by the percentage of identical residues. Standard methods are available for the alignment of sequences, e.g. algorithms of Needleman and Wunsch (J Mol Biol (1970) 48, 443), Smith and Waterman (Adv Appl Math (1981) 2, 482), Pearson and Lipman (Proc Natl Acad Sci USA (1988) 85, 2444), and others. Suitable software is commercially available, e.g. the GCG suite of software (Devereux et al (1984), Nucl Acids Res 12, 387), where alignments can be produced using, for example, GAP or BESTFIT with default parameters, or successors thereof. The Blast algorithm, originally described by Altschul et al (J. Mol. Biol. (1990) 215, 403), but further refined to include gapped alignments (Blast 2), available from various sources such as the EBI, NCBI, will also produce alignments and calculate the % identity between two sequences.

“Specific binding” refers to the binding to substantially only a single antigen.

“FXII/FXIIa” refers to either or both of Factor XII and activated Factor XII (FXIIa). Thus “FXII/FXIIa inhibitor” includes inhibitors of either or both of FXII and FXIIa. Further, anti-FXII/FXIIa antibodies include antibodies that bind to and inhibit either or both of FXII and FXIIa.

“Treating” or “treatment” means the reduction of any symptoms associated with HAE, especially the reduction of the severity and/or frequency of HAE attacks.

“Preventing” or “prevention” means the prevention of any symptoms associated with HAE including the deterioration of the disease.

As explained in more detail in the attached examples, the present inventors have been surprisingly able to show for the first time that the anti-FXII antibodies used in the context of the present invention are very active in reducing the number of attacks in hereditary angioedema (HAE) patients. In fact, the antibodies used in the context of the present invention are able to almost completely prevent such HAE attacks, even when administered subcutaneously. The repeat dosing of the anti-FXII antibodies of the present invention over the time and thereby maintaining an antibody concentration in the blood results in a remarkable reduction of the number of HAE attacks. Consequently, the antibodies used in the context of the present invention represent useful agents for the prevention or treatment of HAE.

Therefore, in one aspect, the present invention relates to an anti-FXII antibody comprising
(i) a V_{H} comprising a CDRH1 comprising a sequence set forth in SEQ ID NO:1; a CDRH2 comprising a sequence set forth in SEQ ID NO:2; and a CDRH3 comprising a sequence set forth in SEQ ID NO:3; and
(ii) a V_{L} comprising a CDRL1 comprising a sequence set forth in SEQ ID NO:4; a CDRL2 comprising a sequence set forth in SEQ ID NO:5; and a CDRL3 comprising a sequence set forth in SEQ ID NO:6;

for use in a method of treating or preventing HAE) in a subject, wherein the antibody is administered subcutaneously to the subject.

The CDR sequences are also given in FIG. 10.

Preferably, the antibody used in the context of the invention binds human Factor XIIa-beta with a K_{D} of better than 10⁻⁷M, more preferably better than 3×10⁻⁸M, more preferably better than 10⁻⁸M, even more preferably better than 3×10⁻⁹M, most preferably 10⁻⁹M or even 5×10⁻¹⁰M.

The antibody or antigen binding fragment thereof can be any isotype, including IgG, IgM, IgE, IgD, or IgA, and any subtype thereof. Preferably, the antibody or antigen binding fragment thereof of the invention is a human IgG or variant thereof, preferably human IgG4 or variant thereof. Methods to switch the type of antibody are well known in the art. The nucleic acid molecule encoding the V_{H} or V_{L} region is isolated, and operatively linked to a nucleic acid sequence encoding a different c_{H} or c_{L}, respectively, from the constant region of a different class of immunoglobulin molecule.

The present disclosure encompasses proteins and/or antibodies described herein comprising a constant region of an antibody. This includes antigen binding fragments of an antibody fused to a Fc.

Sequences of constant regions useful for producing the proteins of the present disclosure may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4.

In one embodiment, the constant region is human isotype IgG4 or a stabilized IgG4 constant region.

In one embodiment, the Fc region of the constant region has a reduced ability to induce effector function, e.g., compared to a native or wild-type human IgG1 or IgG3 Fc region. In one embodiment, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are well known in the art.

In one embodiment, the Fc region is an IgG4 Fc region (i.e., from an IgG4 constant region), e.g., a human IgG4 Fc region. Sequences of suitable IgG4 Fc regions will be apparent to the skilled person and/or available in publicly available databases (e.g., available from National Center for Biotechnology Information).

In one embodiment, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one embodiment, a stabilized IgG4 constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. Sci USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional embodiments of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another embodiment, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another embodiment, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138, 2006; and/or Hezareh J Virol; 75: 12161-12168, 2001).

In another embodiment, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

The present disclosure also contemplates additional modifications to an antibody.

For example, the antibody comprises one or more amino acid substitutions that increase the half-life of the protein. For example, the antibody comprises an Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc (and therefore of Fc region-comprising molecules) into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US2007/0135620 or U.S. Pat. No. 7,083,784.

More preferably, the antibody of the invention is a human IgG1 or human IgG4, engineered for enhanced binding to the human neonatal Fc receptor FcRn at a lower pH, e.g. pH 6, which leads to an increased half-life of the antibody in human serum. Methods to screen for optimal Fc variants for optimizing FcRn binding have been described (e.g. Zalevsky et al (2010) Nature Biotech 28, 157-159).

In a preferred embodiment, the antibody used in the context of the present invention is a germlined antibody as defined above.

Other preferred antibodies or antigen binding fragments thereof of the invention comprise mammalian immunoglobulin constant regions, such as the constant regions of mammalian isotypes such as IgG, IgM, IgE, IgD, or IgA, and any subtype thereof. Preferably, the antibody is a mammalian IgG, including mouse IgG, pig IgG, cow IgG, horse IgG, cat IgG, dog IgG and primate IgG or variants thereof. These antibodies may be chimeric antibodies, where the human variable regions of the invention are combined with the constant region of the immunoglobulin of the selected species. Alternatively, the antibody or antigen binding fragments thereof may be produced by grafting the human CDR regions described herein into the framework residues from an immunoglobulin of the selected species.

Preferably the antibodies or antigen binding fragments thereof of the invention are in their mature form, i.e. without the signal peptide; however, the antibodies or antigen binding fragments thereof including the signal peptides are also included in the invention.

In a further preferred embodiment, the anti-FXII antibody comprises a V_{H} comprising a sequence set forth in SEQ ID NO:7 and a V_{L} comprising a sequence set forth in SEQ ID NO:8. Preferably, the anti-FXII antibody is a germlined antibody.

In a further preferred embodiment, the anti-FXII antibody comprises a heavy chain sequence set forth in SEQ ID NO:9 and a light chain sequence set forth in SEQ ID NO:10. These sequences represent full length heavy and light chains of the CSL312 antibody, which is a germlined antibody as defined above. It is especially included in the present invention that the constant region(s) of these heavy and light chains include the modifications as disclosed above.

The amino acid sequence of this especially preferred antibody is also given in FIG. 10.

It is known in the art that depending on the production method, often the terminal lysine of the heavy chain is cleaved of from at least some of the arms of the antibody. Consequently, the present invention includes both that the heavy chain sequence of the antibody does not contain a terminal lysine as shown in SEQ ID NO:9 and that the heavy chain sequence of the antibody comprises an additional lysine linked to the last amino acid of SEQ ID NO:9, and populations of antibodies comprising uncleaved, partially cleaved and fully cleaved species.

Any discussion of an antibody herein will be understood to include any variants of the antibody produced during manufacturing and/or storage. For example, during manufacturing or storage an antibody can be deamidated (e.g., at an asparagine or a glutamine residue) and/or have altered glycosylation and/or have a glutamine residue converted to pyroglutamine and/or have a N-terminal or C-terminal residue removed or “clipped” and/or have part or all of a signal sequence incompletely processed and, as a consequence, remain at the terminus of the antibody. It is understood that a composition comprising a particular amino acid sequence may be a heterogeneous mixture of the stated or encoded sequence and/or variants of that stated or encoded sequence.

The antibody used in the context of the present invention may be produced by any method well-known in the art. For example, the antibody may be produced by introducing a nucleic acid encoding the antibody into a suitable cell, e.g., a mammalian cell line, such as CHO, HEK293, MDCK, COS, HeLa, or myeloma cell lines such as NS0. Another suitable cell line is an insect cell line for use with a baculovirus, such as SF9 cells, SF21 cells, or HighFive™ cells. Yet another cell is a yeast cell, such as Saccharomyces, e.g. S. cerevisiae, or Pichia pistoris. Bacterial host cells such as E. coli are also possible. Methods for introducing DNA into the respective host cells are well known in the art. For example, when the host cell is a mammalian cell line, techniques such as lipofection or electroporation may be used.

The method of producing the antibody may comprise culturing the host cells, such as the cell line or yeast cell, of the invention under appropriate conditions to express the antibody. The antibody may then be purified. The antibody may be secreted by the host cell, and can then easily be purified from the culture supernatant. Techniques for purifying antibodies are well known in the art, and include techniques such as ammonium sulfate precipitation, size exclusion chromatography, affinity chromatography, ion exchange chromatography and others.

When expressed in E. coli, the antibodies or antigen binding fragments thereof may be produced in inclusion bodies. Methods to isolate inclusion bodies and refold the expressed protein are well known in the art.

Consequently, the present invention also relates to an anti-FXII antibody for use in a method of treating or preventing hereditary angioedema (HAE) in a subject, wherein the antibody is administered subcutaneously to the subject, and wherein the anti-FXII antibody is obtained by introducing a nucleic acid encoding the anti-FXII antibody as disclosed above into a cell, preferably the nucleic acids according to SEQ ID NO: 11 and 12, the anti-FXII antibody is produced in the cell and is subsequently purified.

The nucleic acids according to SEQ ID NO: 11 and 12 encode the polypeptides according to SEQ ID NO:9 and 10.

According to the present invention, the antibody is administered subcutaneously to the subject. Methods for formulating antibodies for a subcutaneous administration are well known in the art and include the preparation of a pharmaceutical composition comprising the antibody.

For example, for the preparation of the pharmaceutical composition for subcutaneous administration, the antibody can be mixed with one or more pharmaceutically acceptable carriers, diluents or excipients. For example, sterile water or physiological saline may be used. Other substances, such as pH buffering solutions, viscosity reducing agents, or stabilizers may also be included.

A wide variety of pharmaceutically acceptable excipients and carriers are known in the art. Such pharmaceutical carriers and excipients as well as suitable pharmaceutical formulations have been amply described in a variety of publications (see for example “Pharmaceutical Formulation Development of Peptides and Proteins”, Frokjaer et al., Taylor & Francis (2000) or “Handbook of Pharmaceutical Excipients”, 3^{rd} edition, Kibbe et al., Pharmaceutical Press (2000) A. Gennaro (2000) “Remington: The Science and Practice of Pharmacy”, 20^{th} edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc). In particular, the pharmaceutical composition comprising the antibody of the invention may be formulated in lyophilized or stable soluble form. The polypeptide may be lyophilized by a variety of procedures known in the art. Lyophilized formulations are reconstituted prior to use by the addition of one or more pharmaceutically acceptable diluents such as sterile water for injection or sterile physiological saline solution.

For subcutaneous administration pharmaceutical compositions comprising the antibody can be administered in dosages and by techniques well known in the art. The amount and timing of the administration will be determined by the treating physician or veterinarian to achieve the desired purposes and should ensure a delivery of a safe and therapeutically effective dose to the blood of the subject to be treated.

In an embodiment, the anti-FXII antibody is administered in an amount to maintain a concentration of the antibody in the blood of at least about 3, 5, 7 or 10 μg/mL, preferably about 5 μg/mL between two subsequent administrations of the antibody.

As it can be taken from the examples and especially from FIG. 5, the administration of the anti-FXII antibody of 75 mg, 200 mg or 600 mg results in blood concentrations of the antibody of at least about 3 μg/mL during one treatment cycle, ie the time period between two administrations of the antibody. Even with such low concentrations of the antibody in the blood, in particular, in the steady state, a remarkable reduction of the number of HAE attacks is observed. In addition, as it can also be seen from FIG. 5, the peak of the concentration of the anti-FXII antibody in the blood after an administration of 75 mg or 200 mg needs not to be higher than about 20 μg/mL.

Consequently, in an embodiment of the invention, the anti-FXII antibody is administered in an amount to achieve a maximal concentration of the antibody in the blood of about 20 μg/mL.

In a further embodiment, the anti-FXII antibody is administered in an amount to reduce the activity of FXII including that of its activated forms to a level observed in healthy subjects.

So, the anti-FXII antibody is administered in an amount to normalize the activity of FXII including that of its activated forms.

In a further aspect, the present invention also relates to an anti-FXII antibody for use in a method of treating or preventing hereditary angioedema (HAE) in a subject, wherein the antibody is administered subcutaneously to the subject and wherein the anti-FXII antibody is administered in an amount to reduce the activity of FXII including that of its activated forms to a level observed in healthy subjects.

According to the present invention modest inhibition of the FXII-mediated kallikrein activity at the end of the dosing cycle in the steady state facilitates efficacious results.

Consequently, in a further embodiment, the anti-FXII antibody is administered in an amount sufficient to inhibit less than about 60%, about 50%, about 40%, or about 30% of the FXII-mediated kallikrein activity between two subsequent administrations of the antibody.

The anti-FXII antibody may be administered at a dosage of about 70 mg to 700 mg, of about 75 mg to 150 mg, of about 150 mg to 250 mg, of about 300 mg to 350 mg, of about 350 mg to 700 mg, about 170 mg to 220 mg, preferably at a dosage of about 75 mg, of about 100 mg, of about 150 mg, of about 170 mg, of about 200 mg, of about 300 mg, of about 340 mg or of about 600 mg, preferably at a dosage of about 100 mg or of about 200 mg.

The anti-FXII antibody may be administered once every 1-3 months, once every 1-2 months, once every month. It may also be administered once every two, three, four, five, six, seven or eight weeks.

According to the invention, the anti-FXII antibody may be administered at a dosage of 70 mg to 700 mg once every 1-3 months, of 70 mg to 700 mg once every 1-2 months, of 70 mg to 700 mg once every 2 months, of 70 mg to 700 mg once every six weeks, of 70 mg to 700 mg once every month, of 75 mg to 150 mg once every 1-3 months, of 75 mg to 150 mg once every 1-2 months, of 75 mg to 150 mg once every 2 months, of 75 mg to 150 mg once every six weeks, of 75 mg to 150 mg once every month, of 150 mg to 250 mg once every 1-2 months, of 150 mg to 250 mg once every 2 months, of 150 mg to 250 mg once every six weeks, of 150 mg to 250 mg once every month, of 170 mg to 220 mg once every 1-2 months, of 170 mg to 220 mg once every 2 months, of 170 mg to 220 mg once every six weeks, of 170 mg to 220 mg once every month, of 75 mg once every 1-2 months, of 75 mg once every 2 months, of 75 mg once every six weeks, of 75 mg once every month, of 100 mg once every 1-2 months, of 100 mg once every 2 months, of 100 mg once every six weeks, of 100 mg once every month, of 200 mg once every 1-2 months, of 200 mg once every 2 months, of 200 mg once every six weeks, of 200 mg once every month.

In a preferred embodiment, the antibody is administered at a dosage of about 150 mg to 250 mg, preferably about 170 mg to 220 mg, more preferably about 200 mg, once every 1-3 months, preferably once every 1-2 months, preferably once every month.

In an alternative preferred embodiment, the antibody is administered at a dosage of about 50 mg to 150 mg, preferably about 70 mg to 130 mg, more preferably about 100 mg, once every 1-3 months, preferably once every 1-2 months, preferably once every month.

In another alternative preferred embodiment, the antibody is administered at a dosage of about 300 mg to 350 mg, preferably about 300 mg or 340 mg, once every 1-3 months, preferably once every two months.

According to a preferred embodiment, the subject is a human subject, preferably a human patient having, suspected of having or at risk for HAE.

According to the invention, the anti-FXII antibody is administered subcutaneously to the subject during the method of treating or preventing HAE. Preferably this includes that the anti-FXII antibody is only administered subcutaneously to the subject. Alternatively, it includes that the method also includes another administration, such as intravenous, intraarterial, intradermal, intraperitoneal, oral, transmucosal, epidural, or intrathecal administration, preferably an intravenous administration.

In one embodiment, the method includes an administration of a loading dose of the anti-FXII antibody. This loading dose may be at the same dosage as the following administrations, or it may be at a higher or lower dosage. Furthermore, said loading dose may be administered subcutaneously, or it may be administered as discussed above, preferably intravenously. The loading dose may be administered at the same time the following administration starts or shortly before it (i.e., within about a week). In case of a subcutaneous administration of the loading dose, such loading dose will preferably be the same amount and given at the same time than the subsequent first dosage. This results in a first dosing which is doubled compared to the subsequent dosages. In case of an intravenous administration of the loading dose, the initial dosage will usually be lower than the subsequent dosages, e.g., about 25%, 50% or 75% of the subsequent dosages. Preferably the loading dose is given shortly before the subsequent dosages.

In a preferred embodiment, the administration of a loading dose is an intravenous administration of the anti-FXII antibody at a dosage of between about 30 mg and 400 mg, preferably between 100 and 300 mg, more preferably 200 mg. For example, in case of a subsequent subcutaneous administration of about 75 mg, the loading dose may be between about 30 mg and 60 mg, in case of a subsequent subcutaneous administration of 100 mg, the loading dose may be between about 40 mg and 70 mg, in case of a subsequent subcutaneous administration of 200 mg, the loading dose may be between about 80 mg and 130 mg, and case of a subsequent subcutaneous administration of about 600 mg, the loading dose may be between about 240 mg and 700 mg.

In a further preferred embodiment, the administration of a loading dose is a subcutaneous administration of the anti-FXII antibody at a dosage of between about 70 mg and 700 mg, preferably between 200 and 500 mg, more preferably 400 mg. For example, in case of a subsequent subcutaneous administration of about 75 mg, the loading dose at the same time may be about 75 mg (i.e. at the first administration a total dose of 150 mg is administered subcutaneously), in case of a subsequent subcutaneous administration of about 100 mg, the loading dose at the same time may be about 100 mg, in case of a subsequent subcutaneous administration of about 200 mg, the loading dose at the same time may be about 200 mg, and case of a subsequent subcutaneous administration of about 600 mg, the loading dose at the same time may be about 600 mg.

In the context of the present invention, and as shown in the examples, the present inventors were able to demonstrate that by the administration of the antibody used in the context of the present invention, the number of HAE attacks can be reduced significantly.

Consequently, in a preferred embodiment of the present invention, the administration of the anti-FXII antibody reduces the risk of an HAE attack, preferably by more than 85%, preferably more than 90% and even more preferably by more than 95% or more than 98%. Preferably, the reduction applies in comparison to non-treated subjects.

In a further aspect, the present invention also relates to a method of treating or preventing hereditary angioedema (HAE) in a subject, wherein said method includes the subcutaneous administration to an anti-FXII antibody to this subject comprising (i) a V_{H} comprising a CDRH1 comprising a sequence set forth in SEQ ID NO:1; a CDRH2 comprising a sequence set forth in SEQ ID NO:2; and a CDRH3 comprising a sequence set forth in SEQ ID NO:3; and (ii) a V_{L} comprising a CDRL1 comprising a sequence set forth in SEQ ID NO:4; a CDRL2 comprising a sequence set forth in SEQ ID NO:5; and a CDRL3 comprising a sequence set forth in SEQ ID NO:6. The anti-FXII antibody is preferably administered to the subject in a therapeutic active amount.

All embodiments disclosed above with respect to the other aspects of the invention also apply to this aspect of the invention.

The invention is further described with the help of the following figures and examples, which are intended to illustrate, but not to limit the present invention.

### BRIEF DESCRIPTION OF THE FIGURES

FIG. 1 explains the contact system comprising FXII, Kallikrein and Bradykinin and the mode of action of the anti-FXII antibody.

FIG. 2 explains the dosing and dose escalation scheme of the phase 1 study as conducted in Example 1.

FIG. 3 shows the blood concentration of the CSL312 after the intravenous (IV) or subcutaneous (SC) administration of the anti-FXII antibody CSL312 in healthy subjects.

FIG. 4 explains the study design of the phase 2 study as conducted in Example 2.

FIG. 5 shows the mean (SD) PK profiles of plasma concentration of the CSL312 antibody (ng/mL) during Treatment Period 1 after administration of the antibody in a concentration of 75 mg (dot), 200 mg (triangle) or 600 mg (square), respectively, between day 63 and day 91 as an example for the steady state (PK population). Note: PK=pharmacokinetic. The PK population consists of all subjects for whom at least 1 measurable concentration of CSL312 was reported. The x-axis denotes the elapsed time in days since first CSL312 administration at day 1.

FIG. 6 shows the mean (SD) PD profiles of FXII mediated kallikrein activity during Treatment Period 1 after administration of the CSL312 antibody in a concentration of 0 mg (Placebo, ring), 75 mg (dot), 200 mg (triangle) or 600 mg (square), respectively, between day 63 and day 91 as an example for the steady state (% Baseline) (PD Population). Note: PD=pharmacodynamics. The PD population consists of all subjects for whom at least 1 PD measurement was reported. The x-axis denotes time since first CSL312 administration at time 0, which corresponds to visit day 1.

FIG. 7 shows the time independent relationship between the FXII mediated kallikrein activity and the CSL312 concentration in the blood.

FIG. 8 gives the mean attack rate after administration of the CSL312 antibody in a concentration of 0 mg (Placebo), 75 mg, 200 mg or 600 mg, respectively.

FIG. 9 shows dosing regimens for the envisaged phase 3 study and the respective predicted attack rates.

FIG. 10 gives the CSL312 heavy and light chain amino acid sequences. The respective CDR sequences are underlined. The C-terminal Lysine of the heavy chain is marked with an asterisk indicating that it is encoded but may be partially or completely removed post translationally.

FIG. 11 explains the study design of the phase 3 study of Example 3.

### KEY TO SEQUENCE LISTING

SEQ ID NO:1 is an amino acid sequence from a CDR1 variable domain of anti-FXII antibody CSL312

SEQ ID NO:2 is an amino acid sequence from a CDR2 variable domain of anti-FXII antibody CSL312

SEQ ID NO:3 is an amino acid sequence from a CDR3 variable domain of anti-FXII antibody CSL312

SEQ ID NO:4 is an amino acid sequence from a CDR1 variable domain of anti-FXII antibody CSL312

SEQ ID NO:5 is an amino acid sequence from a CDR2 variable domain of anti-FXII antibody CSL312

SEQ ID NO:6 is an amino acid sequence from a CDR3 variable domain of anti-FXII antibody CSL312

SEQ ID NO:7 is an amino acid sequence from the heavy region variable domain of anti-FXII antibody CSL312

SEQ ID NO:8 is an amino acid sequence from the light region variable domain of anti-FXII antibody CSL312

SEQ ID NO:9 is an amino acid sequence from the heavy chain variable domain of anti-FXII antibody CSL312

SEQ ID NO:10 is an amino acid sequence from the light chain variable domain of anti-FXII antibody CSL312

SEQ ID NO:11 is a nucleic acid sequence encoding for the heavy chain of anti-FXII antibody CSL312

SEQ ID NO:12 is a nucleic acid sequence encoding for the heavy chain of anti-FXII antibody CSL312

### EXAMPLES

### Example 1

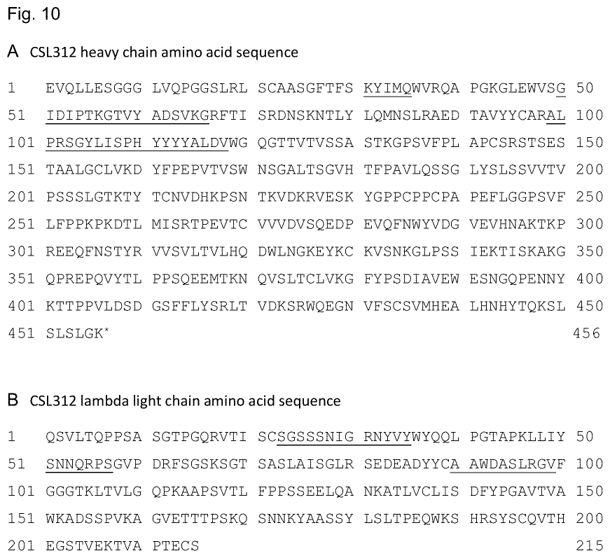

A single-center, randomized, double-blind, placebo-controlled, single ascending dose, phase 1 study was performed to investigate the safety, tolerability, and PK of escalating doses of CSL312 after a single IV infusion or SC injection in healthy subjects. The heavy and the light chain of CSL312 are provided in FIG. 10.

Study Design

CSL312 is a fully human IgG4/lambda recombinant monoclonal antibody which specifically binds to the catalytic domain of activated FXII (FXIIa and βFXIIa) and potently inhibits its catalytic activity. CSL312 inhibits bradykinin (BK) production in vitro and attenuates edema formation in vivo in BK-mediated edema models. CSL312 attenuates expression of inflammatory mediators.

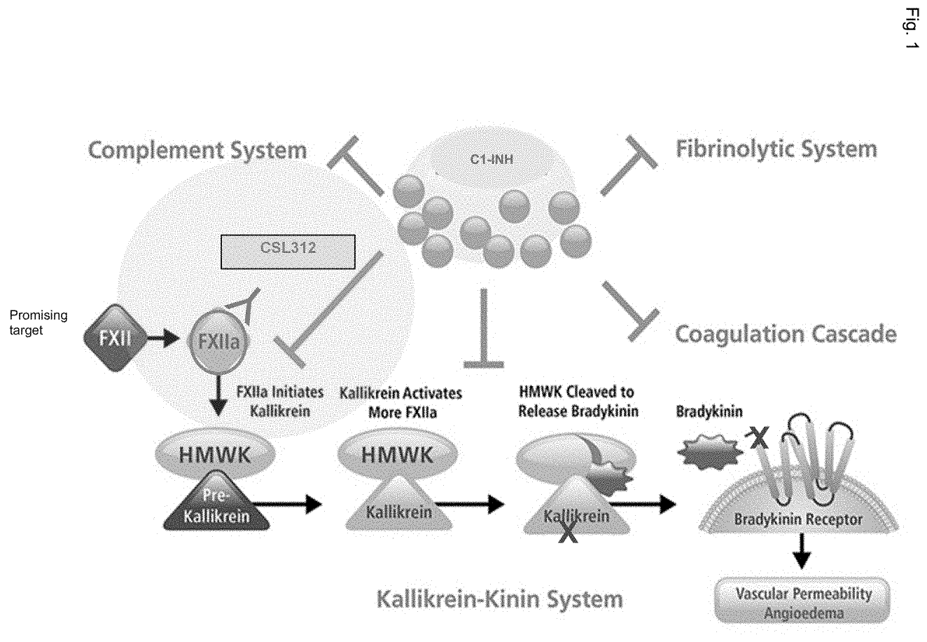

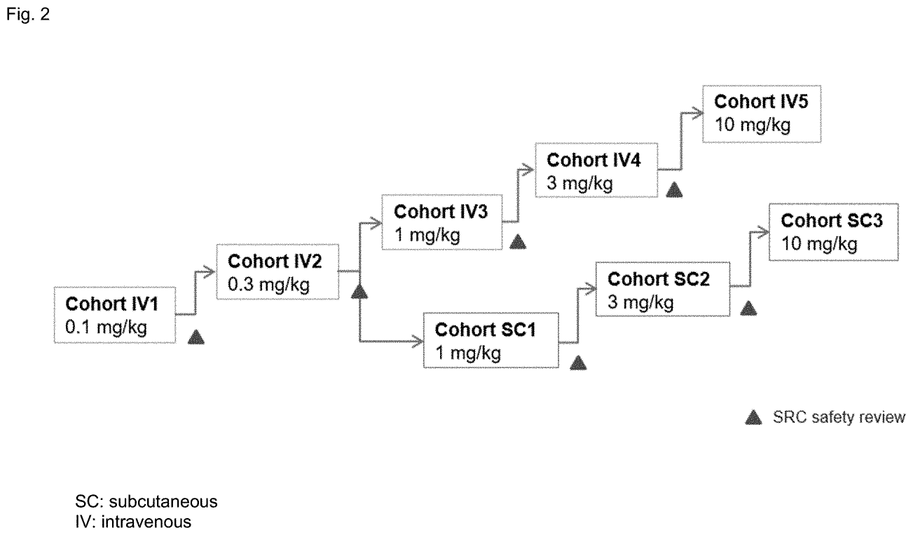

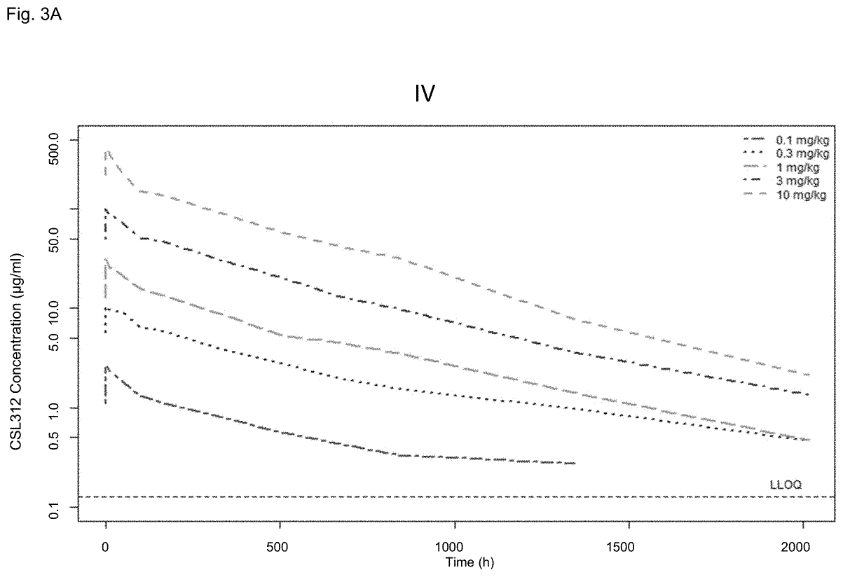

A total of 48 subjects were randomized to 1 of 8 cohorts (5 IV cohorts and 3 SC cohorts) (FIG. 1). Each cohort comprised of 6 subjects (4 active and 2 placebo). Subjects in each of the 5 IV cohorts were administered single CSL312 IV doses of 0.1, 0.3, 1, 3 or 10 mg/kg, or placebo (formulation buffer). Subjects in each of the 3 SC cohorts were administered single CSL312 SC injections of 1, 3 or 10 mg/kg, or placebo (formulation buffer). (FIG. 2: Dosing and Dose Escalation Schema)

Sentinel dosing was implemented for each IV cohort and the first SC cohort. The first 2 enrolled subjects (sentinel subjects) were randomized and received either CSL312 (1 subject) or placebo (1 subject), and were monitored for 48 hours. The principal investigator and medical monitor then assessed safety data from the 48-hour monitoring period. After no safety issues were identified, an additional 4 subjects were randomized and received either CSL312 or placebo (3:1 ratio); dosing of these 4 subjects commenced a minimum of 48 hours after dosing of the 2nd sentinel subject.

Safety Analysis and Results:

All AE summaries were restricted to treatment-emergent AEs (TEAEs) only. Subjects who experienced the same TEAE (in terms of the preferred term) more than once were only counted once for that event in the number of subjects but all occurrences of the same event were counted in the number of events.

Hematology, biochemistry, and coagulation data were summarized at each scheduled visit, with actual values and changes from Baseline. The frequency of the occurrence of anti-drug antibodies (ADAs) by time point were summarized for all subjects who received CSL312. Descriptive summaries were provided for observed values and changes from Baseline for 12-lead electrocardiogram parameters and vital signs assessments.

Overall, 43/48 subjects (89.6%) of subjects experienced at least 1 TEAE. More subjects experienced TEAEs with CSL312 (30/32 subjects [93.8%] with 106 events) than with placebo (13/16 subjects [81.3%] with 50 events). The majority of TEAEs reported after treatment with CSL312 or placebo had a severity grade of Grade 1 (100/106 events [94.3%] with CSL312 and 45/50 events [90.0%] with placebo). Less than a third of all TEAEs were assessed as related to CSL312 or placebo (31/106 events [29.2%] with CSL312 and 14/50 events [28.0%] with placebo). With the exception of 3 TEAEs that were ongoing in 3 subjects who received placebo, all TEAEs had an outcome of recovered or resolved.

No dose dependent trends were seen in TEAE frequency or severity. No deaths, serious AEs, or AEs leading to discontinuation were reported.

Infusion/injection site reactions were reported for a higher proportion of subjects who received CSL312 (18/32 subjects [56.3%] with 21 events) than subjects who received placebo (5/16 subjects [31.3%] with 9 events), primarily due to events reported in the SC cohorts. Overall, in the IV cohorts, the proportion of subjects with infusion site reactions was similar with CSL312 and placebo (30.0% for both treatments). In the SC cohorts, all subjects who received SC CSL312 experienced at least 1 injection site reaction compared with 33.3% of subjects who received SC placebo. All infusion/injection site reactions were Grade 1 and had an outcome of recovered or resolved.

There were no thromboembolic events, bleeding, or anaphylaxis events.

There were no clinically relevant trends in hematology, biochemistry, urinalysis, coagulation, or complement activity results. While abnormal laboratory values were observed in individual subjects, no safety concerns were identified.

No subjects tested positive for anti-CSL312 antibodies at Baseline or at any time point during the study.

No clinically relevant trends were reported for the electrocardiogram or vital signs assessments.

Pharmacokinetics Analysis and Results:

PK parameters and CSL312 plasma concentrations were summarized descriptively by active treatment. All PK parameters were calculated using actual sampling times. Summary statistics for concentration-time data included number of subjects in the analysis population, number of actual observations, and the percentage of below the limit of quantitation (BLQ) values relative to the total number of observations.

Dose proportionality was assessed separately for the IV doses and the SC doses for the PK parameters Cₘₐₓ, AUC_{0-inf}, and AUC₀₋ₜ. Exploratory dose proportionality was analyzed with a power model. Linear proportionality between the PK parameters and dose could be declared if the 90% confidence interval (CI) was within the predefined critical interval of 0.85 to 1.15 for the IV infusions or 0.7 to 1.3 for the SC injections.

The PK parameters AUC_{0-inf} and AUC from time 0 to the last quantifiable time point post-dose (AUC₀₋ₗₐₛₜ) for the subjects who received an IV infusion of CSL312 was compared with the AUC_{0-inf} and AUC₀₋ₗₐₛₜ for the subjects who received an SC injection of CSL312. Comparisons were done between the same doses for IV and SC (i.e., 1 mg/kg IV and 1 mg/kg SC, etc.) as well as between the pooled IV and pooled SC doses using an analysis of variance model.

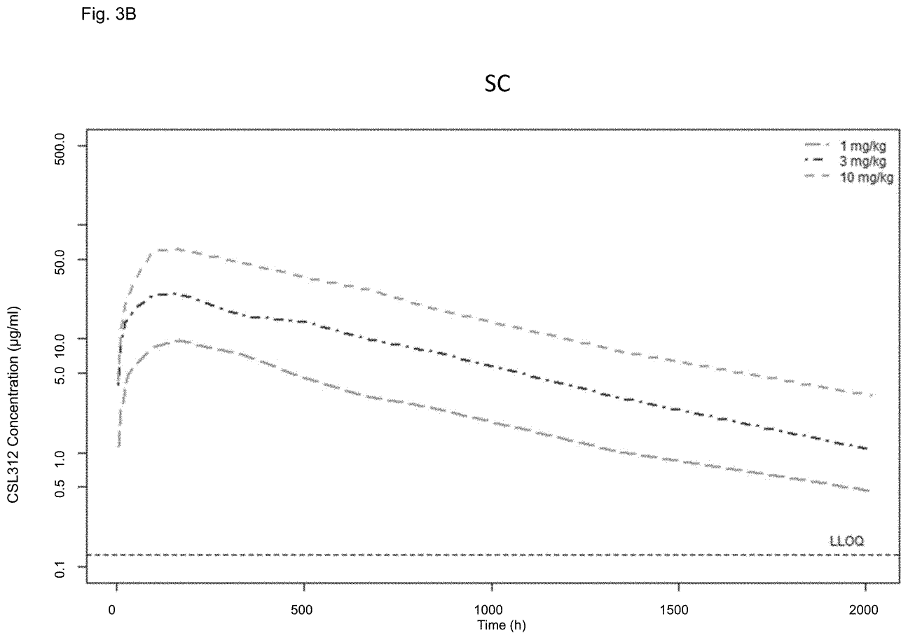

Following single IV infusions of CSL312, the plasma concentrations generally peaked at the end of infusion (at 1 hour), except for the 0.1 mg/kg dose which peaked at approximately 4 hours. Mean t_{1/2} ranged between approximately 14 and 20 days across the IV doses (see FIG. 3A).

Following single SC injections of 1, 3, or 10 mg/kg CSL312, the plasma concentrations peaked at approximately 7 days (168 hours), 5 days (120 hours), and 7 days (168 hours), respectively. Mean t_{1/2} ranged between approximately 18 and 20 days across the SC doses (see FIG. 3B).

Single doses of CSL312 showed a dose dependent increase in CSL312 Cₘₐₓ and AUC when administered as an IV infusion at doses of 0.1, 0.3, 1, 3, and 10 mg/kg or as an SC injection at doses of 1, 3, and 10 mg/kg.

An overall comparison of pooled SC doses versus pooled IV doses estimated bioavailability of dose-normalized AUC_{0-inf} at 49.7%.

In conclusion, CSL312 was safe and well tolerated when administered as a single IV infusion or single SC injection up to 10 mg/kg to healthy male subjects. CSL312 exhibited linear PK when administered as a single IV infusion or SC injection with absolute bioavailability of ˜50% and t_{1/2} of ˜18 days after the SC injection.

### Example 2

A multicenter, randomized, placebo-controlled, parallel-arm, phase 2 study was performed to investigate the clinical efficacy, pharmacokinetics, pharmacodynamics and safety of CSL312 as prophylaxis to prevent hereditary angioedema (HAE) attacks in subjects with C1-INH HAE.

Study Design

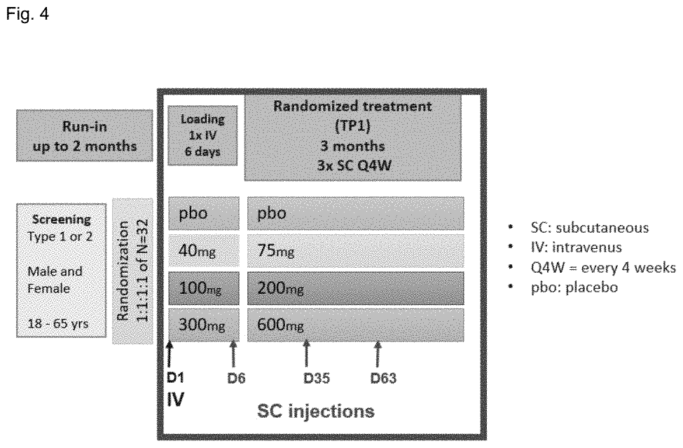

Multiple subcutaneous doses of CSL312 were administered to HAE patients at the following doses: 75 mg 200 mg, or 600 mg. The study consisted of a Screening Period (≤4 weeks), a Run-in Period (≤8 weeks), Treatment Period 1 (˜13 weeks), Treatment Period 2 (˜44 weeks), and a Follow-up Period (˜14 weeks). An overview of the main study design including the Run-in Period and the randomized Treatment Period 1 is presented in FIG. 4.

After Screening, eligible subjects entered into an initial Run-in Period lasting at least 4 and up to 8 weeks to confirm their underlying disease status and to assess their eligibility for participation in Treatment Period 1. Subjects with C1-INH HAE stopped participation in the Run-in Period and began Treatment Period 1 when they met pre-specified criteria, including having experienced ≥2 HAE attacks within a consecutive 4-week period during the Run-in Period.

A total of 32 subjects with C1-INH HAE who were eligible to participate in the blinded Treatment Period 1 were randomly assigned to treatment with one of the following treatment regimens in a blinded manner:
A single loading dose of 40 mg CSL312 intravenous (IV) followed ˜1 week later by 75 mg CSL312 subcutaneously [SC] every 4 weeks [q4wk] for 12 weeks (9 patients);
A single loading dose of 100 mg CSL312 IV followed ˜1 week later by 200 mg CSL312 SC q4wk for 12 weeks (8 patients);
A single loading dose of 300 mg CSL312 IV followed ˜1 week later by 600 mg CSL312 SC q4wk for 12 weeks (7 patients);
A single loading dose of placebo IV followed ˜1 week later by placebo SC q4wk for 12 weeks (8 patients).

All 32 patients completed Treatment Period 1 and began treatment in Treatment Period 2.

Investigators assessed and documented the occurrence of HAE attacks based on data reported by subjects in an electronic diary (eDiary). safety, PK/PD parameters and use of on-demand HAE medication were also assessed.

Subjects who completed the 13 weeks Treatment Period 1 were eligible to participate in Treatment Period 2. Subjects who continued to participate in an open-label treatment period 2 received CSL312 (200 mg or 600 mg) q4wk SC, as assigned. Investigators continue to assess and document the occurrence of HAE attacks based on data reported by subjects in an eDiary. Safety and PK parameters also continue to be assessed. Treatment Period 2 is being conducted in an open-label manner for all subjects.

All subjects, including those who discontinue participation, attended a follow-up visit ˜14 weeks after each subject's final visit in their Treatment Period.

Dose Selection

The dose selection for phase 2 was based on the safety, PK and PD data obtained in the phase 1 single ascending dose study after administration in healthy volunteers (Example 1). The key PD endpoint used for dose selection was FXIIa-mediated kallikrein activity. The inhibitory capacity of CSL312 was studied using biomarker of the kallikrein-kinin system. Kallikrein activity informs how CSL312 contributes to the HAE pathophysiology. Plasma samples were activated ex vivo, mimicking a HAE attack and resulting in FXII-mediated amplification of the kallikrein-kinin pathway. FXIIa cleaves prekallikrein to generate kallikrein whose activity can be measured using chromogenic peptide substrates. It was hypothesized that inhibiting FXIIa-mediated kallikrein activity consistently to a particular % target inhibition is expected to provide protection from HAE attacks. The exact % target FXIIa-mediated kallikrein inhibition to prevent HAE attacks was unknown. A PK/PD model was developed to quantify the relationship between CSL312 plasma concentrations and FXIIa-mediated kallikrein activity in the phase 1 single ascending dose study after administration in healthy volunteers. The modeled relationship showed an increase in inhibition of FXIIa-mediated kallikrein activity with increasing concentrations of CSL312. Based on the relationship between CSL312 plasma concentration and FXIIa-mediated kallikrein activity, the % target inhibition levels that were chosen included ≥30, ≥50, and ≥90% to provide information along the entire spectrum of the curve allowing for a robust assessment of doses in this study. Simulations using the final PK/PD model determined that fixed doses of 75 mg, 200 mg, and 600 mg administered every 4 weeks would result in at least 75% of the patients reaching a % target inhibition of FXIIa-mediated kallikrein activity of ≥30, ≥50, and ≥90%, respectively.

Study Population

To have entered the Run-in Period, subjects must have met all of the following inclusion criteria:
1. Provided written informed consent.
2. Male or female.
3. Aged ≥18 to ≤65 years at the time of providing written informed consent.
4. A Clinical diagnosis of C1-INH HAE, based on the following criteria:
   For C1-INH HAE (type 1):
      Documented clinical history consistent with HAE (subcutaneous or mucosal, non-pruritic swelling episodes without accompanying urticaria).
      C1-INH antigen concentration or functional activity <50% of the lower limit of the reference range, as documented in the subject's medical record.
      C4 antigen concentration below the lower limit of the reference range, as documented in the subject's medical record.
   For C1-INH HAE (type 2):
      Documented clinical history consistent with HAE (subcutaneous or mucosal, non-pruritic swelling episodes without accompanying urticaria).
      C1-INH functional activity <50% of the lower limit of the reference range, as documented in the subject's medical record.
      C4 antigen concentration below the lower limit of the reference range, as documented in the subject's medical record.
5. For subjects with C1-INH HAE: 4 HAE attacks over a consecutive 2-month period during the 3 months before Screening, as documented in the subject's medical record. Note: For subjects taking any prophylactic HAE therapy during the 3 months before Screening, ≥4 HAE attacks may have been documented over any consecutive 2-month period during the 3 months before commencing the prophylactic therapy.
6. Willing to cease the use of C1-INH products, androgens or antifibrinolytics for routine prophylaxis against HAE attacks on the first day of the Run-in Period, after being assessed by the investigator to be able to adequately manage on-demand treatments of HAE attacks without assistance.
7. Investigator believed that the subject understood the nature, scope and possible consequences of the study.

Subjects must not have entered the Run-in Period if they met any of the following exclusion criteria:
1. History of clinically significant arterial or venous thrombosis, or current clinically significant prothrombotic risk (including presence of a central venous access device).
2. History of an uncontrolled, abnormal bleeding event due to a coagulopathy, or a current clinically significant coagulopathy or clinically significant risks for bleeding events.
3. Any pre-planned surgeries during the trial that had an inherent clinically significant risk for thrombotic events or bleeding.
4. Known incurable malignancies at the time of Screening.
5. For subjects with a clinical diagnosis of C1-INH HAE, a clinically significant history of poor response to C1-INH therapy for the management of HAE.
6. Female subjects with C1-INH HAE who started taking or changed dose of any hormonal contraceptive regimen or hormone replacement therapy (i.e., estrogen/progesterone containing products) within 3 months before Screening.
7. Participation in another interventional clinical study during the 30 days before Screening or within 5 half-lives of the final dose of the investigational product administered during the previous interventional study, whichever was longer.
8. Any previous treatment with any monoclonal antibody, recombinant protein bearing an Fc domain, ribonucleic acid (RNA) silencing, or gene transfer technologies.
9. Receiving any other therapy not permitted during the study at the time of Screening.
10. Male or female subject of childbearing potential either not using or not willing to use a highly-effective method of contraception or not sexually abstinent at any time during Treatment Period 1 or Treatment Period 2 and during the Follow-up Period, or not surgically sterile.
11. Intention to become pregnant or to father a child at any time during the study.
12. Pregnant or nursing mother.
13. Known or suspected hypersensitivity to the investigational product or to any excipients of the investigational product.
14. Employee of the study site, or spouse/partner or relative of the investigator or any sub-investigator.
15. Any other issue that, in the opinion of the investigator, would render the subject unsuitable for participation in the study.

Subjects were eligible to exit the Run-in Period and begin Treatment Period 1 if they met the following criteria:
1. Subject participated in the Run-in Period for at least 4 weeks (28 days).
2. For subjects with C1-INH HAE, confirmation of diagnosis by central laboratory testing:
   For subjects with C1-INH HAE (type 1):
      C1-INH antigen concentration or functional activity <50% of the lower limit of the reference range.
      C4 antigen concentration below the lower limit of the reference range.
   For subjects with C1-INH HAE (type 2):
      C1-INH functional activity <50% of the lower limit of the reference range.
      C4 antigen concentration below the lower limit of the reference range.
3. For subjects with C1-INH HAE: the occurrence of ≥2 HAE attacks within any consecutive 4-week period during the Run-in Period.
4. Did not have any clinical abnormalities assessed as clinically significant by the investigator in results of hematology, chemistry, or urinalysis assessments performed during Screening. Note: Subjects with ≥2 times the upper limit of normal for aspartate aminotransferase and/or alanine aminotransferase may have been eligible for participation if there was an explanation for this laboratory result and if the results were not clinically significant.

Study Objective

The primary objective of this study is to evaluate the efficacy of CSL312 in the prevention of HAE attacks in subjects with C1-INH HAE. The primary endpoint is the time-normalized number (per month) of HAE attacks in subjects with C1-INH HAE on treatment with CSL312 or placebo a4 wk during Treatment Period 1.

The secondary objectives of the study are:
To further evaluate the efficacy of CSL312 in subjects with C1-INH HAE.
To evaluate the PK of CSL312 in subjects with C1-INH HAE.
To evaluate the safety and tolerability of CSL312 in subjects with C1-INH HAE.

Safety:

CSL312 was safe and well tolerated at all doses. There were no dose-dependent safety concerns. The percentage of subjects experiencing at least 1 AE during treatment with any dose of CSL312 was similar to placebo. All AEs were nonserious and were assessed as mild or moderate intensity. No subject with C1-INH HAE experienced an SAE (serious adverse event), an AE of special interest (anaphylaxis, thromboembolic event or bleeding event) or an AE leading to discontinuation during blinded treatment with CSL312. No deaths were reported.

Pharmacokinetics, Pharmacodynamics and Efficacy:

All 32 randomized patients (mean age 40 years [range 20-65]; 56% female; 91% white; 94% HAE type 1) completed the Treatment Period 1. Treatment with CSL312 SC every 4 weeks achieved statistical significance in reduction of HAE attack rate compared with placebo. The study also demonstrated clinically meaningful results in the prevention of HAE attacks for secondary endpoint.

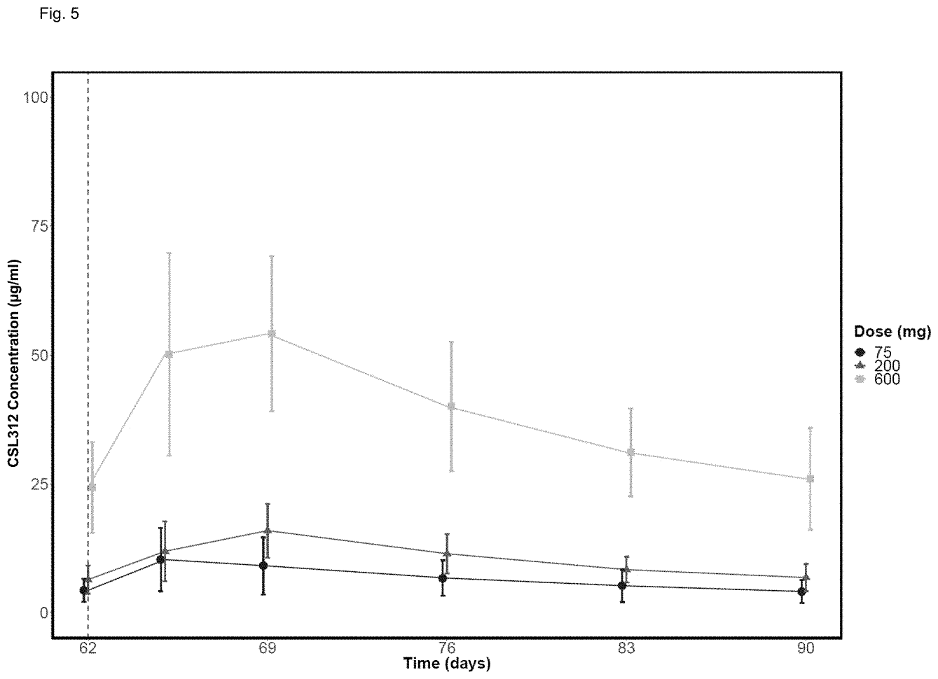

Following a loading dose and three SC administrations the plasma concentration of CSL312 after day 63 peaked about 3 to 7 days after the third SC injection for all three doses. These data for the steady state as well as the dose dependent increase of the mean plasma levels of CSL312 are given in FIG. 5 (graphs in the plot from top to bottom correspond to the regimens as listed from bottom to top).

Table 1 presents a summary of the plasma PK parameters after the last SC administration of CSL312 in Treatment Period 1 (Visit Day 63). After the last SC administration of CSL312 in Treatment Period 1 (Visit Day 63), mean Cₘₐₓ ranged between 10.6 and 56.4 μg/mL. Mean Cₘₐₓ increased approximately 1.5- and 5-fold with a 2.7- and 8-fold increase in dose between the 75 mg and 200 mg and between the 75 mg and 600 mg SC doses of CSL312, respectively. Mean AUC₀₋ₜₐᵤ ranged from 4507 to 26,514 h*μg/mL. Mean AUC₀₋ₜₐᵤ increased approximately 1.6- and 6-fold with a 2.7- and 8-fold increase in dose between the 75 mg and 200 mg and between the 75 mg and 600 mg SC doses of CSL312, respectively. Mean T_{1/2} ranged between approximately 16 and 18 days across doses. Overall, after the last SC administration of CSL312 in Treatment Period 1 (Visit Day 63), CSL312 Cₘₐₓ and AUC increased in a dose-dependent manner.

TABLE 1
Summary of PK plasma parameters of CSL312 by dose,
visit day63, Treatment Period 1 (PK population)

| | 75 mg | 200 mg | 600 mg |
|---|---|---|---|
| | CSL312 | CSL312 | CSL312 |
| | q4wk | q4wk | q4wk |
| | N = 9 | N = 8 | N = 7 |
| Cₘₐₓ (μg/mL) | | | |
| Number of Subjects | 9 | 8 | 7 |
| with Data | | | |
| Mean (SD) | 10.6 (6.09) | 15.9 (5.22) | 56.4 (15.9) |
| Median | 9.11 | 13.9 | 60.2 |
| Min, Max | 4.70, 23.0 | 10.4, 25.9 | 31.3, 75.0 |
| AUC_{0 - tau} (h*μg/mL) | | | |
| Number of Subjects | 9 | 8 | 7 |
| with Data | | | |
| Mean (SD) | 4507 (2424) | 7344 (2488) | 26514 (8151) |
| Median | 3912 | 6146 | 25604 |
| Min, Max | 2354, 10507 | 4558, 12187 | 15630, |
| | | | 39058 |
| Tₘₐₓ (h) | | | |
| Number of Subjects | 9 | 8 | 7 |
| with Data | | | |
| Median | 143.38 | 165.51 | 165.63 |
| Min, Max | 45.37, | 115.52, | 72.42, |
| | 195.58 | 217.50 | 187.57 |
| T_{1/2} (h) | | | |
| Number of Subjects | 7 | 7 | 5 |
| with Data | | | |
| Mean (SD) | 411.72 | 394.01 | 443.46 |
| | (96.96) | (85.64) | (43.99) |
| Median | 445.62 | 379.02 | 447.27 |
| Min, Max | 193, 467 | 296, 515 | 378, 487 |

N = number of subjects assigned to the treatment;
PK = pharmacokinetic;
q4wk = administered every 4 weeks;
Cₘₐₓ = maximum concentration;
max = maximum;
min = minimum;
AUC_{0 - tau} = area under the concentration-time curve in 1 dosing interval;
T_{1/2} = terminal elimination half-life;
Tₘₐₓ = time of maximum concentration.
Note:
The PK population consists of all subjects in the Safety population for whom at least 1 measurable concentration of CSL312 was reported.

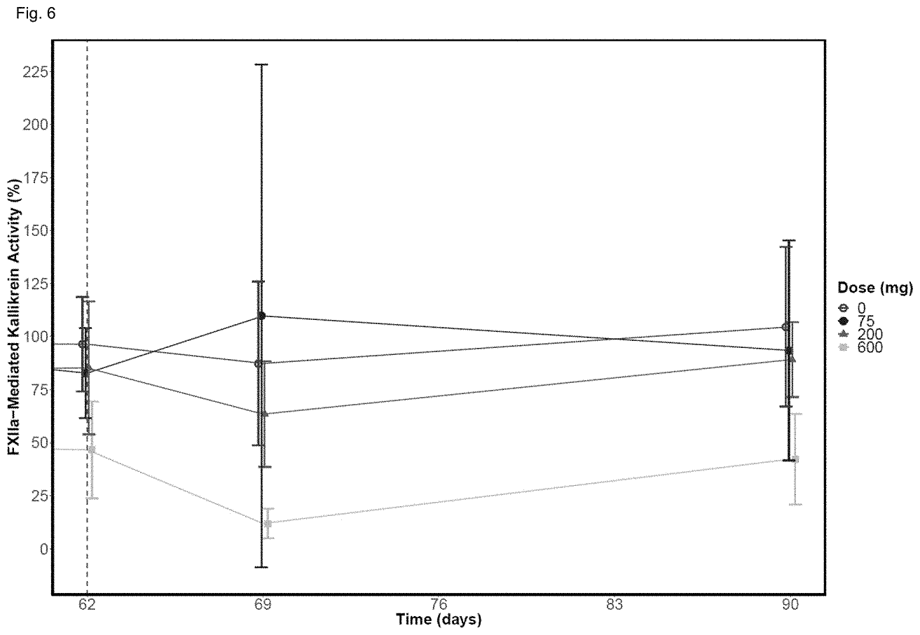

FIG. 6 represents the mean (SD) percent of baseline profiles of FXIIa mediated kallikrein activity at steady state by treatment i.e. after day 63. 100% kallikrein activity is the baseline (kallikrein activity before treatment) of this plot i.e. all the values plotted are their values relative to baseline for each HAE subject. This Figure demonstrates dose dependent inhibition of FXIIa mediated kallikrein activity (graphs in the plot from top to bottom correspond to the regimens as listed from top to bottom).

In general, dose-dependent inhibition of FXIIa-mediated kallikrein activity was observed following administration of CSL312. Mean FXIIa-mediated kallikrein activity was higher in the 75 mg treatment arm at some sampling points compared to placebo. This is likely because of high variability in the results. Near complete inhibition of FXIIa-mediated kallikrein activity was observed at peak concentrations of CSL312 after SC administration of the 600 mg dose.

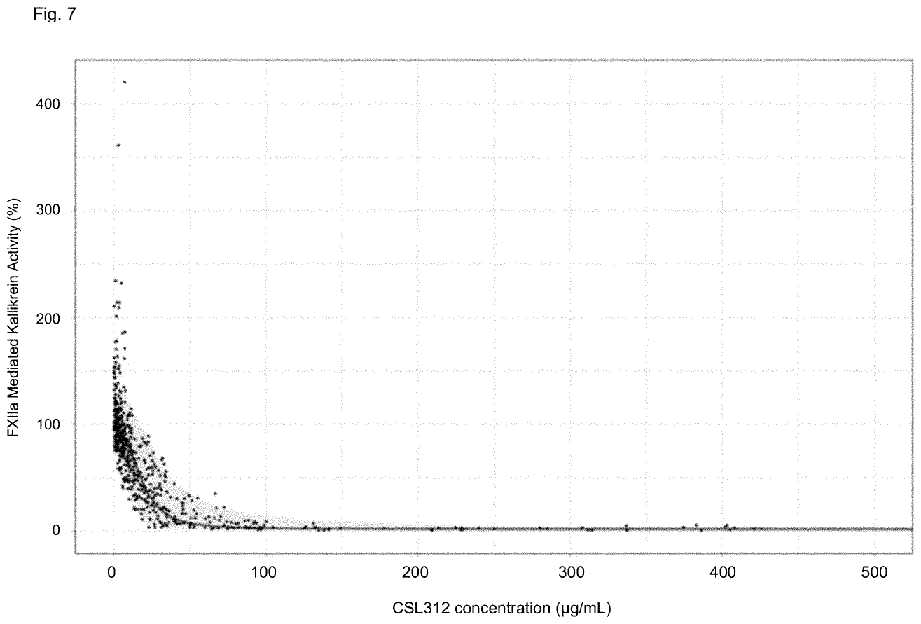

FIG. 7 represents the simulated and observed exposure (CSL312 concentrations in blood)-response (FXIIa mediated kallikrein activity) relationship based on data from healthy and HAE subjects. At CSL312 plasma concentrations of >50 μg/mL the FXIIa mediated kallikrein activity is completely inhibited.

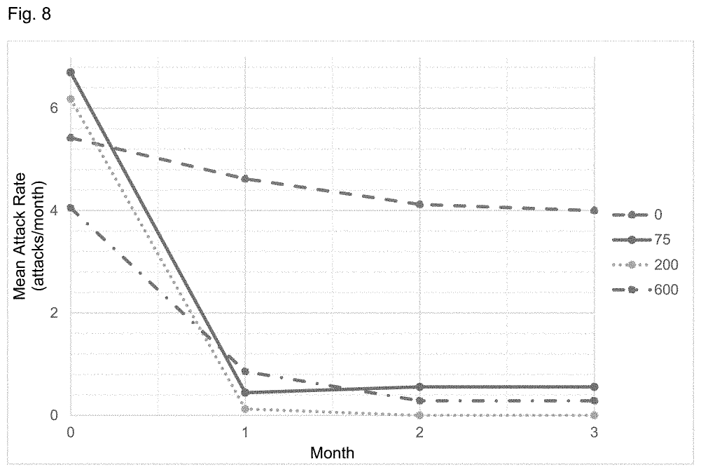

The HAE mean attack rate in time by dose is shown in FIG. 8. It can be seen from these figures and in particular from FIG. 8 that there is no clinically significant difference between the three doses used in phase 2.

The primary efficacy endpoint of the phase 2 study was the time-normalized number of HAE attacks. Treatment with 75 mg, 200 mg or 600 mg CSL312 resulted in a clinically relevant reduction in the time-normalized number of HAE attacks when compared to placebo (Table 2). The mean (SD) time-normalized number of HAE attacks was 4.24 (1.801) in the placebo arm, 0.05 (0.127) in the 200 mg CSL312 treatment arm, and 0.40 (0.514) in the 600 mg CSL312 treatment arm. The mean reduction in the time-normalized number of HAE attacks was 98.94% with 200 mg CSL312 and 90.50% with 600 mg CSL312, relative to placebo. Treatment with 75 mg CSL312 was also assessed, but no formal statistical comparisons between 75 mg CSL312 and placebo were conducted. However, summary statistics demonstrate efficacy after treatment with this dose. The mean (SD) time-normalized number of HAE attacks was 0.48 (1.057). The mean reduction in the time-normalized number of HAE attacks was 88.68% with 75 mg CSL312, relative to placebo.

The secondary endpoints were responder subjects, HAE attack-free subjects, HAE attacks, HAE attacks treated with on-demand HAE medication, and CSL312 PK in plasma (Cₘₐₓ, Tₘₐₓ, Tw₂, AUC, see Table 1).

TABLE 2
Time-normalized number of HAE attacks (mean attack rate in
number of attacks/month) in subjects with C1-INH HAE
randomized to blinded treatment in Treatment
Period 1

| | | 75 mg | 200 mg | 600 mg |
|---|---|---|---|---|
| | Placebo | CSL312 | CSL312 | CSL312 |
| | q4wk | q4wk | q4wk | q4wk |
| | N = 8 | N = 9 | N = 8 | N = 7 |
| Time- | | | | |
| normalized | | | | |
| Number | | | | |
| of HAE | | | | |
| Attacks | | | | |
| per Month | | | | |
| Mean (SD) | 4.24 | 0.48 | 0.05 | 0.40 |
| | (1.801) | (1.057) | (0.127) | (0.514) |
| Median | 4.61 | 0.00 | 0.00 | 0.34 |
| (Min, Max) | (1.40, 7.16) | (0.00, 3.26) | (0.00, 0.36) | (0.00, 1.40) |
| 95% | 2.74, 5.75 | −0.33, 1.29 | −0.06, 0.15 | −0.07, 0.88 |
| confidence | | | | |
| interval | | | | |

N = Number of evaluable subjects

Analyses of the percentage of subjects with a ≥50%, ≥70%, or ≥90% reduction in the time-normalized number of HAE attacks (i.e., responders) with CSL312 relative to the Run-in Period were conducted (Table 3). The percentages of responders were higher during treatment with CSL312 compared to treatment with placebo:
The number and percentage of responders with a 50% reduction in HAE attacks were 0 subjects with placebo, 9/9 (100.0%) with 75 mg, 8/8 (100.0%) with 200 mg, and 6/7 (85.7%) with 600 mg.
The number and percentage of responders with a 70% reduction in HAE attacks were 0 subjects with placebo, 8/9 (88.9%) with 75 mg, 8/8 (100.0%) with 200 mg, and 6/7 (85.7%) with 600 mg.
The number and percentage of responders with a 90% reduction in HAE attacks were 0 subjects with placebo, 8/9 (88.9%) with 75 mg, 8/8 (100.0%) with 200 mg, and 4/7 (57.1%) with 600 mg.

Note that analysis of the percentage of responders with a 30% reduction in the time-normalized number of HAE attacks with CSL312 relative to the Run-in Period was also conducted but was not a part of the topline results and is therefore not presented here. Results from this responder analysis were consistent with the results presented above.

TABLE 3
Reduction relative to Run-in Period in the
time-normalized number of HAE attacks
per months occurring in subjects with C1-INH HAE
randomized to blinded treatment in Treatment Period 1

| | | 75 mg | 200 mg | 600 mg |
|---|---|---|---|---|
| | Placebo | CSL312 | CSL312 | CSL312 |
| | q4wk | q4wk | q4wk | q4wk |
| | N = 8 | N = 9 | N = 8 | N = 7 |
| ≥50% Reduction | 0 | 9 (100) | 8 (100) | 6 (85.7) |
| Responder, n (%) | | | | |
| ≥70% Reduction | 0 | 8 (88.9) | 8 (100) | 6 (85.7) |
| Responder, n (%) | | | | |
| ≥90% Reduction | 0 | 8 (88.9) | 8 (100) | 4 (57.1) |
| Responder, n (%) | | | | |

N = Number of evaluable subjects;
n = number of responder subjects
Subjects with a reduction of ≥50%, ≥70%, or ≥90%, respectively, are classified as responders

The mean percent reduction in the time-normalized number of HAE attacks during treatment with CSL312, as compared with placebo, was 88.68% with 75 mg, 98.94% with 200 mg, and 90.50% with 600 mg. In contrast, there was no substantial reduction in the time-normalized number of HAE attacks during treatment with placebo when compared to the Run-in Period (within group comparison). The mean reduction in the time-normalized number of HAE attacks was 9.76% with placebo, relative to the Run-in Period.

Of the 24 subjects randomized to treatment with any dose of CSL312, 15 subjects were HAE attack-free during the efficacy evaluation period. Of these 15 HAE attack-free subjects, 5/9 subjects (55.6% [95% CI: 26.67, 81.12]) were HAE attack-free with 75 mg CSL312, 7/8 subjects (87.5% [95% CI: 52.91, 97.76]) were HAE attack-free with 200 mg CSL312, and 3/7 subjects (42.9% [95% CI: 15.82, 74.95]) were HAE attack-free with 600 mg CSL312. No subjects treated with placebo were HAE attack-free during the efficacy evaluation period.

Subjects who were treated with CSL312 and who were not HAE attack-free during Treatment Period 1 had HAE attack-free periods until the first attack of between 1.7 and 5.1 weeks with 75 mg (4 subjects), and between 1.3 and 9.9 weeks with 600 mg (4 subjects). The single subjects who was not HAE attack-free with 200 mg had 2 HAE attacks with an HAE attack-free period of 2.3 weeks.

In conclusion, blinded treatment CSL312 was safe and well tolerated when administered as a single IV infusion followed by three SC injections every four weeks up to 600 mg to patients with C1-INH HAE.

Subjects with C1-INH HAE who participated in the study were randomized to blinded treatment with placebo, or 75 mg, 200 mg, or 600 mg CSL312 SC q4wk. The results demonstrate that CSL312 was safe and effective for the prevention of HAE attacks in this study population.

Treatment with 75 mg, 200 mg or 600 mg CSL312 SC q4wk resulted in a clinically relevant reduction in the time-normalized number of HAE attacks when compared to placebo. Of the 24 subjects randomized to treatment with any dose of CSL312, 15 subjects were HAE attack-free during the efficacy evaluation period, including 5/9 (55.6%) subjects who were treated with 75 mg CSL312, 7/8 (87.5%) subjects who were treated with 200 mg CSL312 and 3/7 (42.9%) subjects who were treated with 600 mg CSL312. No subjects who were treated with placebo were HAE attack-free during the same evaluation period.

The results demonstrate that CSL312 exhibited dose-dependent PK after SC administration in Treatment Period 1, with a T_{1/2} of ˜17 days. A concentration-dependent inhibition of FXIIa-mediated kallikrein activity was observed following SC administration of CSL312 in Treatment Period 1.

It could be demonstrated compared to state of the art methods of prevention of HAE attacks (based on elevation of C1-INH protein levels to normal or thorough maximum inhibition of kallikrein or specific bradykinin receptor 2 blockage) that this new method prevents HAE attacks by normalizing the kallikrein activity due to partial FXIIa activity inhibition in HAE patients is very efficient.

Preliminary Study Design of Phase 3

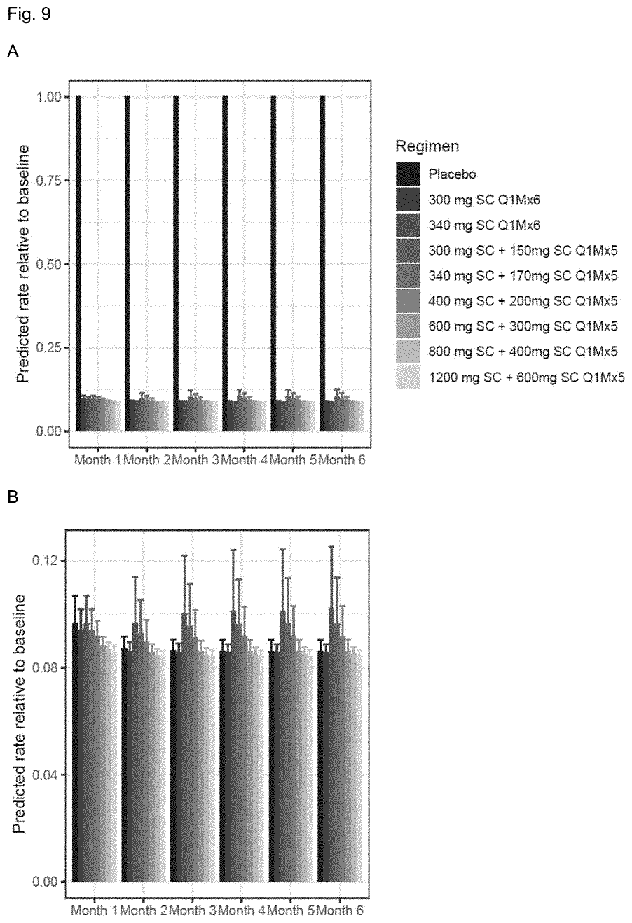

HAE patients are more vulnerable to contact activation than healthy subjects as evidenced by episodic swelling. The clinical efficacy, pharmacokinetics, and safety of CSL312 as prophylaxis to prevent HAE attacks was assessed to determine which dosing regimen to use for a follow-up study to evaluate and confirm the efficacy of CSL312. FIG. 9 represents hypothetical scenarios for the predicted HAE attack rates to administer CSL312 with different SC doses given on a monthly basis and some of them including a SC loading dose (FIG. 9A represents the treatment effect of the selected dose regimen versus placebo; FIG. 9B highlights the difference in attack rates for the selected dosing regimens; bars in the plot from left to right for each time period correspond to the regimens as listed from top to bottom). No significant difference can be seen in efficacy at selected monthly doses for a treatment period of 6 months.

CSL312 plasma concentrations below about 20 μg/mL were associated with partial kallikrein activity inhibition and revealed a clinically meaning prophylactic effect thereby supporting a low dose hypothesis. Thus, continually maintaining CSL312 drug levels above about 5 or 10 μg/mL prevent the over-activation of kallikrein in HAE patients and thus preventing HAE attacks.

### Example 3

Study Overview

This is a multicenter, double-blind, randomized, placebo-controlled, parallel-arm phase 3 study to investigate the clinical efficacy and safety of subcutaneously administered CSL312 as prophylaxis to prevent HAE attacks in subjects with C1-INH HAE type 1 or type 2.

Potential Risks

The following risks have not been observed in the development program of CSL312, but are potential risks based on the drug class and/or the mode of action:

Thromboembolic Events and Bleeding: By blocking FXIIa with CSL312, there may be a potential risk of bleeding or thromboembolic events (TEEs) due to altered hemostasis, unstable clot formation, or impaired clot breakdown. In addition, because of the pharmacological action of CSL312, a prolongation of aPTT is expected to be observed in a dose-dependent manner. Clinical experience with CSL312 in healthy volunteers in the phase 1 study and patients with HAE in the ongoing phase 2 study did not show an effect on either prothrombin time or abnormal bleeding. This is consistent with the observation that patients who have congenital deficiency of FXII do not exhibit a bleeding phenotype, despite having a prolonged aPTT. In addition, nonclinical studies in mice and rabbits showed no impairment in hemostasis after inhibition of FXIIa. Subjects will be monitored carefully for any signs of bleeding or thrombosis during the study.

Severe Hypersensitivity/Anaphylactic-type Reactions: Administration of therapeutic proteins including monoclonal antibodies such as CSL312 is potentially associated with the risk of hypersensitivity and anaphylactic reactions, some of which can be serious and life-threatening. Appropriate precautions will be taken when CSL312 is administered at the study site, with vigilant monitoring for potential severe hypersensitivity and anaphylactic reactions. Administration of CSL312, at least the first 2 to 3 doses, will be performed at the site under medical supervision with immediate access to emergency equipment and medication for the treatment of severe hypersensitivity adverse reactions including and anaphylaxis.

Immunogenicity (anti-drug antibodies): All protein therapeutics are potentially immunogenic. Because CSL312 is a protein, it has the potential to cause the development of neutralizing and non-neutralizing anti-drug antibodies. Subjects will be monitored for the development of immunogenicity throughout the study.

In both the phase 1 study (Example 1) and TP1 of the phase 2 study (Example 2), no severe adverse events (SAEs) were reported. Additionally, no adverse events of special interest (AESIs) were reported in the phase 2 study. There were no dose dependent safety concerns in either study.

Given the potential benefit of CSL312 in patients with COVID-19, the favorable safety data from the phase 1 study and the ongoing phase 2 study, the associated benefit-risk assessment is considered acceptable.

Primary Objective and Endpoint of Study

The primary objective of the study is to evaluate the efficacy of SC administration of CSL312 as prophylaxis to prevent HAE attacks in subjects with HAE. Time-normalized number of HAE attacks during treatment from day 1 through day 182 is the primary endpoint. This is assessed by time-normalized number of HAE attacks (per month and annualized) in subjects treated once a month with either CSL312 (active arm) or placebo (placebo arm) during the period from day 1 through day 182 (6 months).

Secondary Objectives and Endpoints of Study

The secondary objectives of the study are:

1. To characterize the clinical efficacy of SC CSL312 in the prophylactic treatment of HAE

2. To evaluate the safety of SC CSL312 in the prophylactic treatment of HAE

TABLE 4
Secondary Endpoints

| Secondary | | |
|---|---|---|
| Objectives | Endpoints | Summary Measures |
| 1 | The reduction | The percentage reduction |
| | in the attack | (at least ≥50% ≥70%, |
| | rate during | ≥90 or equal to 100% |
| | the Treatment | [attack free]) in the time- |
| | Period compared | normalized number of |
| | to the Run- | HAE attacks in subjects |
| | in Period | treated once a month |
| | | with either CSL312 or |
| | | placebo during the period |
| | | from day 1 through day |
| | | 182 (6 months) compared |
| | | to the run-in period, as |
| | | well as for the first 3-month |
| | | time period and for |
| | | the second 3-month time |
| | | period of the active and |
| | | placebo arms compared |
| | | to the run-in period |
| 1 | The time- | The time-normalized |
| | normalized number | number (per month and |
| | of HAE attacks | annualized) of HAE attacks |
| | requiring on- | requiring on-demand |
| | demand treatment | treatment in subjects |
| | | treated once a month with |
| | | either CSL312 or placebo |
| | | during the period from |
| | | day 1 through day 182 (6 |
| | | months), as well as for |
| | | the first 3-month time |
| | | period and for the second 3- |
| | | month time period of the |
| | | active and placebo arms |
| 1 | Time-normalized | The time-normalized |
| | number of | number (per month and |
| | moderate and/or | annualized) of moderate |
| | severe HAE | and/or severe HAE |
| | attacks | attacks in subjects treated |
| | | once a month with |
| | | either CSL312 or placebo |
| | | during the period from |
| | | day 1 through day 182 (6 |
| | | months), as well as for |
| | | the first 3-month time |
| | | period and for the second 3- |
| | | month time period of the |
| | | active and placebo arms |
| 1 | Time-normalized | The time-normalized number |
| | number of | of HAE attacks (per |
| | HAE attacks at | month and annualized) in |
| | various time | subjects treated once |
| | points during | monthly with either CSL312 |
| | the treatment | or placebo during the |
| | period | first 3-month time period |
| | | and the second 3-month |
| | | time period of CSL312 |
| | | and placebo. |
| | | The percentage reduction |
| | | will be calculated for |
| | | the time-normalized |
| | | number of HAE attacks |
| | | between the active arm |
| | | and the placebo arm for |
| | | the 6-month treatment |
| | | period, as well as for the |
| | | first 3 months and the |
| | | second 3 months of the |
| | | treatment period. |
| 1 | Subject's Global | Comparison of the |
| | Assessment | distribution of responses to |
| | of Response | therapy between CSL312 |
| | to Therapy | and placebo at the end |
| | (SGART) | of the Treatment Period |
| | | (day 182 or day 91 if |
| | | discontinuation occurs |
| | | before day 182) based on |
| | | the proportions of |
| | | subjects with a “excellent, |
| | | good, fair, poor or none” |
| | | response to therapy |
| 2 | • AEs | The number and |
| | • AESIs | percentage of subjects |
| | • SAEs | experiencing the |
| | • CSL312 | specified safety events on |
| | induced anti- | treatment with CSL312 |
| | CSL312 antibodies | or placebo during the |
| | • Clinically | entire Treatment Period |
| | significant | until follow-up or final |
| | abnormalities | visit. |
| | in laboratory | |
| | assessments | |
| | (ie, laboratory | |
| | abnormalities | |
| | reported as | |
| | AEs). | |

Exploratory Objectives and Endpoints

The exploratory objective of this study is to further evaluate the efficacy, pharmacokinetic (PK)/pharmacodynamics (PD), and quality of life (QoL) associated with the use of CSL312 in subjects with HAE.

Exploratory endpoints include the following:

1. The time to first attack after Day 1 and after Day 15.

2. CSL312 concentrations at scheduled time points.

3. FXII concentration and FXIIa-mediated kallikrein activity at scheduled time points.

4. Subject reported outcome measures:
Angioedema Quality of Life (AE-QoL)
EuroQoL-Group 5-Dimension 5-Level (EQ-5D-5L)
Work Productivity and Activity Impairment: General Health (WPAI:GH).

5. Investigator's Global Assessment of Response to Therapy (IGART).

Study Design

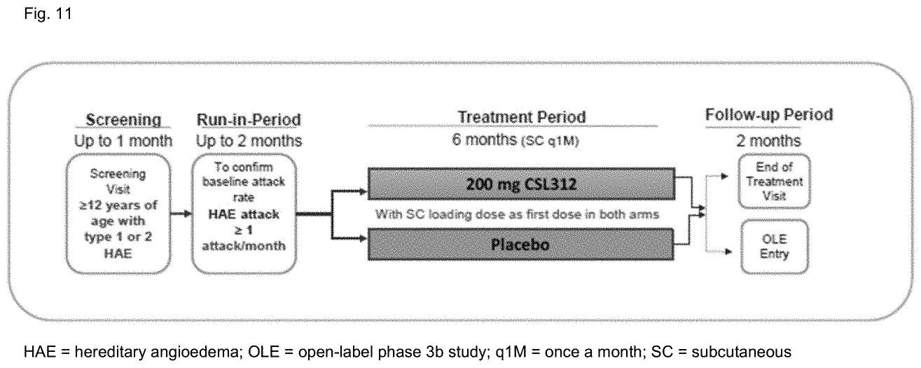

This is a multicenter, double-blind, randomized, placebo-controlled, parallel-arm, phase 3 study to investigate the efficacy and safety of a single dose of SC CSL312 administered once monthly as prophylaxis to prevent HAE attacks in adolescent (12 to 17 years, inclusive) and adult subjects with C1-INH HAE type 1 and type 2. As shown in FIG. 11, the study consists of a Screening Period (up to 1 month), a Run-in Period (up to 2 months) for confirmation of disease activity and determination of subjects' baseline HAE attack rate, 1 Treatment Period (6 months) for confirmation of the safety and efficacy of the 200 mg CSL312 dose, and either a 2-month Follow-up Period (i.e., 3 months after last investigational product administration of investigational product) or entry into the open-label Phase 3b Study.

Screening: Following informed consent, subjects will undergo a Screening Period of up to 1 month to determine eligibility for enrollment into the study. Screened subjects who meet all the inclusion criteria and none of the exclusion criteria will enter the Run-in Period.

Run-in Period: After Screening, eligible subjects will enter the Run-in Period lasting at least 1 month and up to 2 months to confirm their underlying disease status and to assess their eligibility for participation in the Treatment Period. The first day of the Run-in Period may occur on the same day as Screening.

Subjects must complete at least 1 month of the Run-in Period. Additionally, subjects must experience at least 2 HAE attacks during the Run-In period to be eligible to enter the Treatment Period. Subjects who experience at least 2 attacks during the required first month of the Run-in Period may enter the Treatment period. Subjects who do not experience an HAE attack during the first month of the Run-in Period will remain in the Run-in Period for up to an additional month during which time they would be required to experience at least 2 attacks to be eligible to enter the Treatment Period and randomization.

Subjects are not permitted to use routine prophylaxis to prevent HAE attacks during the Run-in Period; however, subjects may use on-demand HAE therapy to treat HAE attacks if that medication has previously been shown to be effective.

Subjects who do not meet the minimum HAE attack rate during the Run-in Period or are otherwise determined to be ineligible due to Screening assessments, will be considered Run-in failures and will not be allowed to be rescreened for participation in the study.

Treatment Period: Subjects meeting the eligibility criteria will enter the Treatment Period after the Run-in Period.

Eligible subjects will be randomized 3:2 to either the CSL312 active arm or the placebo arm. The duration of the Treatment Period is 6 months. Randomization will take age (≤17 years, >17 years) and, for adults, baseline attack rate observed during the Run-in Period (1 to <3 attacks/month, and 3 attacks/month) into account.

Follow-Up Period/Open-label Phase 3b Study Entry:

Subjects who successfully complete the current phase 3 study may have the option to roll over into an open-label phase 3b study (OLE). Subjects who choose not to participate in the OLE study are required to complete the follow-up visit (day 242, which is approximately 3 months after the last dose of investigational product). For subjects who choose to participate in the OLE study, assessments collected on day 182 will be used to fulfill applicable assessments for day 1 of the OLE study.

Dose and Dosing Regimen

The investigational products in this study are 200 mg CSL312 and placebo.

Subjects randomized to the active arm will receive CSL312 SC once a month for 6 months. The first dose of CSL312 will be a 400 mg loading dose administered subcutaneously on the same day as 2 separate injections at the study site (i.e., month 1). Subsequent doses of CSL312 will be 200 mg administered SC once monthly for 5 consecutive months (i.e., months 2 through 6).

Subjects randomized to the placebo arm will receive volume-matched placebo once monthly for 6 months. The first dose of placebo in the placebo arm will be volume-matched placebo administered SC as 2 separate injections (i.e., month 1). Subjects will then receive volume-matched placebo SC once a month for 5 consecutive months (i.e., months 2 through 6).

The proposed dose of 200 mg was selected based on the efficacy and safety observed in TP1 of the phase 2 study (Example 2), CSL312 PK, inhibition of FXIIa-mediated kallikrein activity, and exposure-response (E-R) modeling.

The 200 mg dose administered once every 28 days (±3 days) was highly effective across various efficacy endpoints and had a favorable safety profile. In addition, the 200 mg dose resulted in ˜50% inhibition of FXIIa-mediated kallikrein activity.

To support phase 3 dose selection, an E-R model was used to simulate HAE attack rates over a wide range of CSL312 concentrations that would be expected after different dosing regimens. Based on the E-R model, the estimated daily average concentrations to achieve 50, 75, and 90% relative attack risk reduction in the baseline attack rate were 1.4, 3.3, and 7.8 μg/mL, respectively. The median predicted minimum daily average CSL312 concentrations at steady-state following 200 mg SC once a month regimen corresponds to the 90% relative attack risk reduction in baseline attack rate in 73% of patients.

Additionally, the E-R model showed a cumulative effect of CSL312 concentration is evidenced in the reduction in the expected number of HAE attacks per month. The 200 mg SC once a month regimen is predicted to reduce the mean attack rate by approximately 91% compared to placebo. Increasing the dose beyond 200 mg is not predicted to result in significant further reductions in HAE attacks.

Finally, the exposures at the 200 mg SC dose administered monthly are not expected to cause aPTT prolongation in the majority of subjects in the phase 3 study.

Based on all the factors taken into consideration in selecting a dose, the 200 mg of CSL312 SC administered once monthly is expected to achieve clinically meaningful treatment effect and optimal benefit/risk ratio in subjects with C1-INH HAE type 1 and type 2.

Eligibility Criteria

The study population will be selected on the basis of the inclusion and exclusion criteria described in the sections below. Each subject should meet all of the inclusion criteria and none of the exclusion criteria for this study. Subject eligibility should be reviewed and documented by an appropriately medically qualified member of the investigator's study team before subjects are included in the study.

Inclusion Criteria

To be enrolled and randomized into the study, subjects must meet all of the following inclusion criteria:
1. Capable of providing written informed consent and willing and able to adhere to all protocol requirements and/or the subject's parent(s) or legally acceptable representative(s) capable of providing written informed consent/assent as appropriate.
2. Male or female.
3. Aged ≥12 years at the time of providing written informed consent or assent for minors.
4. Diagnosed with clinically confirmed C1-INH HAE:
   a. Documented clinical history consistent with HAE (subcutaneous or mucosal, nonpruritic swelling episodes without accompanying urticaria), and
   b. C1-INH antigen and/or functional activity s 50% of normal as documented in the subject's medical record, and
   c. C4 antigen concentration below the lower limit of the reference range as documented in the subject's medical record.
5. Experienced 3 HAE attacks during the 3 months before screening, as documented in the subject's medical record.
   Note: For subjects taking any prophylactic HAE therapy during the 3 months before screening, 3 HAE attacks may be documented over 3 consecutive months before commencing the prophylactic therapy.

Exclusion Criteria

Subjects must not be enrolled into the study if they meet any of the following exclusion criteria:
1. Concomitant diagnosis of another form of angioedema, such as idiopathic or acquired angioedema, recurrent angioedema associated with urticarial or HAE type 3.
2. Any preplanned major surgeries or procedures during the clinical study.
3. For adult subjects: Use of C1-INH products, androgens, antifibrinolytics or other small molecule medications for routine prophylaxis against HAE attacks within 2 weeks prior to the Run-in Period.
4. For adolescent subjects 12 to 17 years of age, inclusive: Use of long-term prophylactic therapy for HAE before Screening.
5. Use of monoclonal antibodies such as lanadelumab (Takhzyro®) within 3 months prior to the Run-in Period.
6. Use of estrogen-containing medications with systemic absorption (e.g., oral contraceptive or hormonal replacement therapy), angiotensin-converting enzyme (ACE) inhibitor within 4 weeks prior to the Run-in Period, or currently receiving a therapy not permitted during the study.
7. Participation in another interventional clinical study during the 30 days before screening or within 5 half-lives of the final dose of the investigational product administered during the previous interventional study, whichever is longer.
8. Known or suspected hypersensitivity to monoclonal antibody therapy or hypersensitivity to the investigational product or to any excipients of the investigational product.
9. Subject has any condition that in the judgement of the investigator or CSL, may compromise their safety or compliance, impede successful conduct of the study, interfere with interpretation of the results or would otherwise render the subject unsuitable for participation in the study, e.g., clinically significant bleeding due to coagulopathy, thrombotic disorder, significant illnesses or major comorbidities.
10. Previously administered CSL312 in another interventional clinical study.
11. Intention to become pregnant or to father a child at any time during the study.
12. Female of childbearing potential or male subjects who are fertile and sexually active either not using or not willing to use an acceptable method of contraception to avoid pregnancy during the study and for 30 days after receipt of the last dose of investigational product.

Note: All female subjects are assumed to be of childbearing potential except:
Subjects aged >60 years.
Subjects aged 45 to 60 years (inclusive) with amenorrhea for ≥1 year with documented evidence of follicle-stimulating hormone level >30 IU/L. If the follicle-stimulating hormone value is not available before randomization, a urine pregnancy test is required.
Subjects who are surgically sterile for at least 3 months before providing informed consent.
Note: All male subjects are assumed fertile except subjects who are surgically sterile for at least 3 months before providing informed consent
13. Pregnant, breastfeeding, or not willing to cease breastfeeding.
14. Involved in the planning and/or conduct of the study.

Criteria for entry into Treatment Period

Subjects will be eligible to exit the Run-in Period and enter the Treatment Period if they meet all the following criteria:
1. Participated in the Run-in Period for at least 1 month.
2. Experienced at least an average of 1 HAE attack per month during the Run-in Period (e.g., experienced a total of at least 2 HAE attacks).
3. Does not have laboratory clinical abnormalities assessed as clinically significant by the investigator in results of hematology, chemistry, or urinalysis assessments.
4. C1-INH functional activity and antigen, and C4 antigen concentration levels have been verified prior to randomization.

Note: Subjects with 2 times the upper limit of normal for aspartate aminotransferase and/or alanine aminotransferase may be eligible for participation if there is an explanation for this laboratory result and if the results are not clinically significant.

Study Assessments

Time windows for all assessments are detailed in Table 5.

TABLE 5
Time Windows for Assessments

| | Time window |
|---|---|
| | (relative to |
| | scheduled visit/ |
| Visit/Procedure | procedure) |
| Screening | Not applicable |
| Run-in Period: visit days | ±4 days |
| 15, 30, 45, and 60 | |
| Treatment Period: visit | ±4 days |
| days 31, 61, 91, 121, 151, 182 | |
| Follow-up: Visit day 242 | −14 days |
| Vital signs, physical | Pre-injection |
| examination, and pregnancy test | on same day |

Demographics and Safety Assessments

Subject demographics and safety assessments (including some laboratory assessments) will be conducted during this study.

Pharmacokinetic and Pharmacodynamic Assessments

Plasma samples will be collected during the study for assessment of CSL312 concentration (Pharmacokinetics Evaluations) and FXII concentration and FXIIa mediated kallikrein activity (Pharmacodynamics Evaluations).

Efficacy Assessments

Hereditary angioedema attacks that are confirmed by investigator or designee will be used for the efficacy analysis and will be recorded on the electronic case report form (eCRF). All HAE symptoms reported by the subject will be displayed in a by-subject listing. The investigator will review the symptom(s) reported by the subjects. The investigator will confirm if the symptom(s) represent an HAE attack and, if not an HAE attack, then document the symptom(s) as an AE in the eCRF. A prodromal symptom by itself or use of on-demand medication alone should not be considered as an attack.

At each study visit and phone contact during the Run-in Period, the investigator or designee will review the subject's electronic diary (eDiary) entries. The investigator will consider all available medical information and may ask clarifying questions to assist in their confirmation of HAE attacks.

The following information will be documented in the subject eDiary:
Date and time of HAE symptom onset
Date and time of HAE symptom resolution (i.e., subject no longer experiencing symptoms of the attack)
Location of HAE symptom(s)
Confirmation of interference of symptom(s) with the subject's daily activities
If on-demand medication was used to treat HAE symptoms:
   Name of medication
   Date and time of administration
Confirmation of medical assistance received for the HAE symptoms

The investigator will confirm additional details with the subject related to the symptoms:
Location of HAE symptom(s)
Start/end date/time of symptom(s)
Dose(s) of on demand medication(s) used
Route(s) of Administration of on demand medication(s) used
Self-administered on demand medication(s)? (yes/no)
Administration of on demand medication(s) at a study site, home or emergency room
Type of medical assistance or intervention provided by a healthcare professional during HAE symptoms, including hospitalization or emergency department visits
Severity of the attack (based on degree of interference in daily activities, and whether or not the use of on demand medication and/or medical assistance was needed)

Efficacy Analyses

The primary endpoint “time-normalized number of HAE attacks per month during treatment from Day 1 through Day 182” is calculated per subject as:

[the number of HAE attacks/length of subject treatment in days]*30.4375

where the length of subject treatment is calculated as:

[the date of Study Visit Day 182 or the date of study discontinuation [whatever is first]−the date of Study Visits Day 1+1].

To test for a difference in the primary efficacy endpoint between CSL312 and placebo, a comparison of the time-normalized numbers of HAE attacks in the 6 months of the active arm and in the 6-month placebo arm period will be performed by using a two-sided Wilcoxon Test (alpha=5%).

The time-normalized number per month and per year of HAE attacks will be summarized descriptively for the 6 months of the active arm and the 6-month placebo arm period by median and mean with corresponding 95% confidence intervals (CIs) by treatment.

As a sensitivity analysis, the time-normalized number of HAE attacks will be compared for the 6 months of the active arm and the 6 months of placebo arm using a Poisson Regression model. The time-normalized number of HAE attacks of the Run-in Period and age as covariates and the logarithm of the length of subject treatment as an offset variable will be included. The model will account for overdispersion.

The secondary efficacy endpoint of the percentage reduction in the time-normalized number of HAE attacks is calculated within a subject as:

100*[1−(time-normalized number of HAE attacks per month during treatment/time-normalized number of HAE attacks per month during Run-in)]

for the entire 6-months of the active arm and for the 6-month placebo arm period and will be tested via a two-sided Wilcoxon Test using the individual percentage reduction between treatment groups.

The number and percentage of responders and non-responders will be presented with corresponding 95% CIs. A subject is classified as a responder if the percentage reduction in HAE attacks is ≥50%. In addition, the number and percentage of subjects with percentage reductions of ≥70%, and ≥90% will be presented with corresponding 95% CIs.

The number and percentage of subjects with a percentage reduction of 100%, i.e., who do not experience a HAE attack and so are attack-free, will be presented and summarized with corresponding 95% CI for the 6-month active arm period and for the 6-month placebo arm period, a Fisher-Test will be performed to asses for differences between treatments.

The percentage reduction in the time-normalized number of HAE attacks for the 6-months of the active arm will also be calculated as percentage reduction compared to the 6-month of the placebo arm (between subjects) as

100*[1−(median time-normalized number of HAE attacks per month during 6 months of the active arm/median time-normalized number of HAE attacks per month during 6-months placebo arm period)]

and will be tested as exploratory via a two-sided Wilcoxon Test using the individual percentage reduction between treatment groups.

The secondary efficacy endpoint of time-normalized number of HAE attacks per month requiring on-demand treatment is calculated as:

100*[1−(number of HAE attacks requiring on-demand treatment during treatment/length of subject treatment in days)]*30.4375

An HAE attack requiring on-demand treatment is defined as an attack for which the date of administration of an on-demand treatment is between the start (including) and end date (including) of a HAE attack. Differences between the 6-months of the active arm and the 6-month placebo arm period will be tested in an exploratory manner via a two-sided Wilcoxon Test.

For the analysis of the time-normalized number of moderate and/or severe HAE attacks, an analogue calculation will be done using all HAE attacks classified as moderate or severe.

Safety Analyses

Adverse events with a start date and time occurring after the first administration of the study drug will be considered treatment-emergent adverse events (TEAEs). Adverse events with missing or partial start date or time will also be considered TEAEs following the worst-case principle unless the partial data clearly indicates that the AE started before first administration date and time. Treatment-emergent AEs occurring until the Follow-up Visit will be summarized. Only TEAEs will be included in analysis, although all AEs will be listed.

Pharmacokinetics Analyses

The PK analysis will be performed using the PK population. Plasma concentrations of CSL312 will be listed by individual subjects and will be summarized by nominal time points. Individual and mean CSL312 plasma concentration versus time will be plotted on linear and semi-logarithmic scales. Plasma CSL312 concentrations will be summarized with descriptive statistics: mean, SD, percent coefficient of variation, median, minimum, maximum, and first and third quartiles for continuous variables, geometric mean and its respective 90% CI.

Pharmacodynamic Analyses

Pharmacodynamic data will be summarized using the PD population. FXIIa-mediated kallikrein activity and FXII concentration will be assessed for the pharmacodynamics of CSL312 as described above. FXIIa-mediated kallikrein activity and FXII concentration will be listed by individual subject and summarized by nominal time point and treatment.

1-18. (canceled)

19. A method of treating hereditary angioedema (HAE) in a subject in need thereof, comprising administering to the subject a therapeutically effective amount of an anti-FXII antibody, wherein the anti-FXII antibody comprises:
a) a V_{H} comprising a CDRH1 comprising a sequence set forth in SEQ ID NO:1; a CDRH2 comprising a sequence set forth in SEQ ID NO:2; and a CDRH3 comprising a sequence set forth in SEQ ID NO:3; and
b) a V_{L} comprising a CDRL1 comprising a sequence set forth in SEQ ID NO:4; a CDRL2 comprising a sequence set forth in SEQ ID NO:5; and a CDRL3 comprising a sequence set forth in SEQ ID NO:6.

20. The method of claim 19, wherein the anti-FXII antibody comprises a V_{H} comprising a sequence set forth in SEQ ID NO: 7 and a V_{L} comprising a sequence set forth in SEQ ID NO:8.

21. The method of claim 19, wherein the anti-FXII antibody is an IgG.

22. The method of claim 21, wherein the anti-FXII antibody is an IgG4.

23. The method of claim 21, wherein the antibody comprises a mutation to proline at position 228 of the hinge region according to the EU numbering system.

24. The method of claim 19, wherein the anti-FXII antibody comprises a heavy chain sequence set forth in SEQ ID NO:9 and a light chain sequence set forth in SEQ ID NO:10.

25. The method of claim 24, wherein the heavy chain comprises an additional lysine linked to the last amino acid of SEQ ID NO:9.

26. The method of claim 19, wherein the anti-FXII antibody is administered in an amount to maintain a concentration of the antibody of at least 5 μg/ml between two subsequent administrations of the antibody.

27. The method of claim 19, wherein the antibody is administered at a dosage of 70 mg to 700 mg once every 1-3 months.

28. The method of claim 19, wherein the antibody is administered at a dosage of 150 mg to 250 mg.

29. The method of claim 19, wherein the antibody is administered at a dosage of 50 mg to 150 mg.

30. The method of claim 19, wherein the antibody is administered every 1-2 months.

31. The method of claim 19, wherein the subject is a human patient having, suspected of having, or at risk for HAE.

32. The method of claim 19, wherein the method includes an administration of a loading dose of the anti-FXII antibody.

33. The method of claim 32, wherein the administration of a loading dose is an intravenous administration of the anti-FXII antibody at a dosage of between 30 mg and 400 mg.

34. The method of claim 32, wherein the administration of a loading dose is a subcutaneous administration of the anti-FXII antibody at a dosage of between 70 mg and 700 mg.

35. The method of claim 19, wherein the anti-FXII antibody is only administered subcutaneously to the subject.

36. The method of claim 19, wherein the administration of the anti-FXII antibody reduces the risk of an HAE attack by at least 85%.

37. The method of claim 19, wherein the anti-FXII antibody is administered in an amount sufficient to inhibit less than 60% of the FXII-mediated kallikrein activity between two subsequent administrations of the antibody.

38. The method of claim 19, wherein the anti-FXII antibody is administered in an amount sufficient to inhibit less than 50% of the FXII-mediated kallikrein activity between two subsequent administrations of the antibody.


SEQUENCE LISTING (12 sequences)

SEQ ID NO: 1 - protein, 5 aa, Artificial Sequence
  CSL312 CDRH1 variable domain
KYIMQ

SEQ ID NO: 2 - protein, 17 aa, Artificial Sequence
  CSL312 CDRH2 variable domain
GIDIPTKGTVYADSVKG

SEQ ID NO: 3 - protein, 20 aa, Artificial Sequence
  CSL312 CDRH3 variable domain
ALPRSGYLISPHYYYYALDV

SEQ ID NO: 4 - protein, 13 aa, Artificial Sequence
  CSL312 CDRL1 variable domain
SGSSSNIGRNYVY

SEQ ID NO: 5 - protein, 7 aa, Artificial Sequence
  CSL312 CDRL2 variable domain
SNNQRPS

SEQ ID NO: 6 - protein, 10 aa, Artificial Sequence
  CSL312 CDRL3 variable domain
AAWDASLRGV

SEQ ID NO: 7 - protein, 129 aa, Artificial Sequence
  CSL312 heavy region variable domain
EVQLLESGGGLVQPGGSLRLSCAASGFTFSKYIMQWVRQAPGKGLEWVSGIDIPTKGTVYADSVKGRFTISRDNSKNTLYLQMNSLRAEDTAVYYCARALPRSGYLISPHYYYYALDVWGQGTTVTVSS

SEQ ID NO: 8 - protein, 109 aa, Artificial Sequence
  CSL312 light region variable domain
QSVLTQPPSASGTPGQRVTISCSGSSSNIGRNYVYWYQQLPGTAPKLLIYSNNQRPSGVPDRFSGSKSGTSASLAISGLRSEDEADYYCAAWDASLRGVFGGGTKLTVL

SEQ ID NO: 9 - protein, 455 aa, Artificial Sequence
  CSL312 heavy chain variable domain
EVQLLESGGGLVQPGGSLRLSCAASGFTFSKYIMQWVRQAPGKGLEWVSGIDIPTKGTVYADSVKGRFTISRDNSKNTLYLQMNSLRAEDTAVYYCARALPRSGYLISPHYYYYALDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 215 aa, Artificial Sequence
  CSL312 light chain variable domain
QSVLTQPPSASGTPGQRVTISCSGSSSNIGRNYVYWYQQLPGTAPKLLIYSNNQRPSGVPDRFSGSKSGTSASLAISGLRSEDEADYYCAAWDASLRGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - DNA, 1368 bp, Artificial Sequence
  CSL312 heavy chain encoding sequence
gaggtgcagctgctggaatctggcggcggactggtgcagcctggcggctccctgagactgtcttgcgccgcctccggcttcaccttctccaagtacatcatgcagtgggtccgacaggctcctggcaagggcctggaatgggtgtccggcatcgacatccccaccaagggcaccgtgtacgccgactccgtgaagggccggtttaccatctcccgggacaactccaagaacaccctgtacctgcagatgaactccctgcgggccgaggacaccgccgtgtactactgtgccagagccctgcctcggagcggctacctgatctccccccactactactactatgccctggacgtgtggggccagggcaccaccgtgaccgtgtcctctgcctccaccaagggcccaagcgtgttccccctggccccctgctccagaagcaccagcgagagcacagccgccctgggctgcctggtgaaggactacttccccgagcccgtgaccgtgtcctggaacagcggagccctgaccagcggcgtgcacaccttccccgccgtgctgcagagcagcggcctgtacagcctgagcagcgtggtgaccgtgcccagcagcagcctgggcaccaagacctacacctgtaacgtggaccacaagcccagcaacaccaaggtggacaagagggtggagagcaagtacggcccaccctgccccccctgcccagcccccgagttcctgggcggacccagcgtgttcctgttcccccccaagcccaaggacaccctgatgatcagcagaacccccgaggtgacctgtgtggtggtggacgtgtcccaggaggaccccgaggtccagttcaactggtacgtggacggcgtggaggtgcacaacgccaagaccaagcccagagaggagcagtttaacagcacctaccgggtggtgtccgtgctgaccgtgctgcaccaggactggctgaacggcaaagagtacaagtgtaaggtctccaacaagggcctgccaagcagcatcgaaaagaccatcagcaaggccaagggccagcctagagagccccaggtctacaccctgccacccagccaagaggagatgaccaagaaccaggtgtccctgacctgtctggtgaagggcttctacccaagcgacatcgccgtggagtgggagagcaacggccagcccgagaacaactacaagaccacccccccagtgctggacagcgacggcagcttcttcctgtacagcaggctgaccgtggacaagtccagatggcaggagggcaacgtctttagctgctccgtgatgcacgaggccctgcacaaccactacacccagaagagcctgagcctgtccctgggcaag

SEQ ID NO: 12 - DNA, 645 bp, Artificial Sequence
  CSL312 light chain encoding sequence
cagtctgtgctgacccagcctccttccgcctctggcacccctggccagagagtgaccatctcctgctccggctcctcctccaacatcggccggaactacgtgtactggtatcagcagctgcccggcaccgcccccaagctgctgatctactccaacaaccagcggccctccggcgtgcccgaccggttctctggctccaagtctggcacctccgcctccctggccatctccggcctgagatctgaggacgaggccgactactactgcgccgcctgggacgcttctctgcggggagtgtttggcggaggcaccaagctgaccgtcctaggtcaacccaaggccgctcccagcgtgaccctgttcccccccagcagcgaggagctgcaggccaacaaggccaccctggtgtgtctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgaccccagagcagtggaagagccacaggagctacagctgccaggtcacccacgagggcagcaccgtggaaaagaccgtggccccaaccgagtgctcc